(19) United States
(12) Patent Application Publication
(10) Pub. No.: US 2011/0288123 A1
(43) Pub. Date: Nov. 24, 2011

(54) FORMULATIONS FOR THE TREATMENT OF ACUTE HERPES ZOSTER PAIN

(75) Inventors: Edward T. Kisak, San Diego, CA (US); R. Dominic King-Smith, San Diego, CA (US); Bradley S. Galer, West Chester, PA (US); John M. Newsam, La Jolla, CA (US); Nadir Buyuktimkin, San Diago, CA (US); Servet Buyuktimkin, San diego, CA (US); Jagat Singh, Toronto (CA)

(73) Assignee: NUVO RESEARCH INC., Mississauga, ON (CA)

(21) Appl. No.: 13/127,470

(22) PCT Filed: Nov. 5, 2009
(86) PCT No.: PCT/US2009/063414

§ 371 Date: Jul. 22, 2011

Related U.S. Application Data
(60) Provisional application No. 61/112,123, filed on Nov. 6, 2008.

(51) Int. Cl. A61K 31/47; A61K 31/167; A61P 29/00; A61K 31/24; A61P 23/00; A61K 31/245; A61K 31/445

(52) U.S. Cl. 514/312; 514/535; 514/626; 514/330; 514/317; 514/534; 514/536

(57) ABSTRACT

The present invention provides compositions and methods that are useful for treatment of pain associated with acute herpes zoster. The aqueous compositions are non-stinging and non-irritating.

### FIELD OF THE INVENTION

The present invention relates generally to compositions and methods for treating pain associated with acute herpes zoster.

### BACKGROUND OF THE INVENTION

Acute herpes zoster (“AHZ”) is commonly known as “shingles.” Each year, it afflicts approximately 1 million Americans (see, Weaver B A., J Am Osteopath Assoc. 2007 March; 107(3 Suppl 1):S2-7; Website of Center for Disease Control) and 1.8 million Europeans within the 25 EU countries (see, Johnson R W, Rice A S. Pain. 2007 March; 128(1-2):3-5. Epub 2006 Dec. 11). The vast majority of these patients are middle-aged or elderly, with at least half over 50 years of age. The major risk factor for developing AHZ is age (over 50 years old), although compromised immune function due either to immune disorder or medication such as that used in chemotherapy, can also increase risk.

Initially, patients may have a “prodrome” where they experience pain and discomfort in the area where the rash will eventually develop. The rash of AHZ typically is maculopapular with vesicles that may last 2-4 weeks until healing. The AHZ rash is always unilateral (one-sided) along a dermatome, most commonly in the chest region (thoracic) and on the forehead (trigeminal), though AHZ can occur anywhere on the body.

The pain accompanying AHZ can be throbbing, stabbing, burning, or lancinating in character (see, Weaver B A, 2007) and has been shown to be moderate to severe in intensity within 72 hours of rash onset (see, Dworkin R H, Nagasako E M, Johnson R W, Griffin D R. Pain. 2001 October; 94(1):113-9). Over 80% of AHZ patients experience allodynia. It is believed that the most of the pain is not a direct consequence of the rash, but instead is a result of viral inflammation of the nerves. The vast majority of patients with AHZ will have a self-limited pain condition, with less than 50% having some pain and perhaps less than 10% having “clinically meaningful pain” at 6 months (see, Thyregod H G, Rowbotham M C, Peters M, Possehn J, Berro M, Petersen K L. Pain. 2007 March; 128(1-2):148-56. Epub 2006 Oct. 27). According to a US government patient handout, “about 1 person in 5” will develop chronic postherpetic neuralgia (“PHN”) pain (see, Website of Center for Disease Control). Risk factors for developing PHN include old age and severe pain during AHZ.

The current recommended treatment for AHZ is initiation of antiviral treatment within 48 to 72 hours of disease onset which can shorten the duration of symptoms and perhaps lower the risk of chronic postherpertic neuralgia (see, Landow K. Postgrad Med. 2000 June; 107(7):107-8, 113-4, 117-8). The oral antiviral agents prescribed for treating AHZ are famciclovir (Famvir®), valacyclovir hydrochloride (Valtrex®), and acyclovir (Zovirax®). Seven days of therapy at full dose is recommended.

Currently, there are no FDA-approved topical medications to treat the pain associated with AHZ. Patients with AHZ are prescribed oral non-steroidal anti-inflammatory drugs (“NSAID”) and oral mixed opioids (hydrocodone/acetaminophen and oxycodone/acetaminophen) and, less commonly, oral neuropathic pain medication, such as antidepressants and anticonvulsants. These medications are mediocre at best at alleviating the pain and all have potential significant systemic side-effects.

Another option to treat the pain is a sympathetic nerve block. However, this is an invasive and potentially dangerous procedure and has serious side-effects.

U.S. Pat. No. 5,411,738 and the related publications of Rowbotham, M. C. et al. (Ann Neurol, 1995, 37:246-253) and Rowbotham, M. C. and Fields, H. L. (Pain, 1989, 38; 287-301) describe topical formulations that contain lidocaine for the relief of pain in an individual suffering from herpes zoster or post-herpetic neuralgia. However, the formulations taught in U.S. Pat. No. 5,411,738 and the related publications are not suitable for acute herpes zoster. As mentioned above, acute herpes zoster is associated with skin rashes and open skin lesions, and thus non-stinging and low irritancy topical formulations are required. The formulations described in these publications have high concentrations of irritating and stinging ingredients and thus may result in stinging, pain, and discomfort upon application to the zoster lesioned skin. For instance, the lidocaine gels and patches described in Rowbotham, M. C. et al. (1995) and Rowbotham, M. C. and Fields, H. L (1989) contain very high amounts of propylene glycol, which is known to cause stinging (see, U.S. Pat. Nos. 3,928,556 and 6,958,159). The FDA has approved a lidocaine patch marked under the tradename Lidoderm™ for the treatment of postherpetic neuralgia, a neuropathic pain condition that occurs in a small fraction of patients after the herpes zoster rash has healed. The Lidoderm™ patch contains lidocaine base and dihydroxylaluminum, aminoacetate, disodium edetate, gelatin, glycerin, kaolin, methylparaben, polyacrylic acid, polyvinyl alcohol, propylene glycol propylparaben, sodium carboxymethylcellulose, sodium polyacrylate, D-sorbitol, tarartic acid and urea. According to the FDA approved package insert, this product should only be applied to intact skin (See DailyMed database from the National Institute of Health). Furthermore, removing (i.e. pealing off) a medicated patch would also result in pain and discomfort, and thus is not preferable.

In view of the above, there is an unmet need for a topical formulation to relieve or treat pain in an individual suffering from acute herpes zoster without causing additional pain and discomfort with its use. The present invention satisfies this important clinical need as well as other needs.

### BRIEF SUMMARY OF THE INVENTION

Acute herpes zoster is associated with skin rashes and lesions, and thus a non-stinging and low irritancy topical formulation is strongly preferred for treatment. As such, in one embodiment, the present invention provides an aqueous pharmaceutical composition, the composition comprising, or consisting essentially of, or consisting of:
a) a topically acting anesthetic active ingredient in a subanesthetic amount;
b) 0% to 5% w/w of a lower alkanol;
c) a molecular penetration enhancer; and
d) a carrier.

The aqueous pharmaceutical composition is useful for the management of pain associated with an acute herpes zoster infection. The formulation may be made sterile or bacteriostatic for safe application to skin that is compromised by AHZ. In certain aspects, the formulation is sprayable, and as such, it is easy to cover a wide area of the skin, or alternatively, a more localized, limited area of skin.

In another embodiment, the present invention provides a method for alleviating pain associated with, for example, an acute herpes zoster infection, the method comprising:
applying to an affected area an aqueous composition comprising, or consisting essentially of, or consisting of
a) a topically acting anesthetic active ingredient in a subanesthetic amount;
b) 0% to 5% w/w of a lower alkanol;
c) a molecular penetration enhancer; and
d) a carrier, to alleviate pain.
   In yet another aspect, the present invention provides a use of an aqueous non-stinging and non-irritating pharmaceutical composition, said composition comprising, or consisting essentially of, or consisting of:
a) a topically acting anesthetic active ingredient in a subanesthetic amount;
b) 0% to 5% of a lower alkanol;
c) a molecular penetration enhancer; and
d) a carrier, in the manufacture of a medicament for the treatment of acute Herpes zoster.

These and other embodiments will become more apparent when read with the accompanying figures and detailed description which follows.

### BRIEF DESCRIPTION OF THE DRAWINGS

FIG. 1 provides a schematic of an accumulated dose of lidocaine using molecular penetration enhancers in Vehicle 1 at infinite dose application.

FIG. 2 provides a schematic of an accumulated dose of lidocaine using molecular penetration enhancers in Vehicle 1 at infinite dose application.

FIG. 3 provides a schematic of an accumulated dose of lidocaine using molecular penetration enhancers in Vehicle 1 at infinite dose application.

FIG. 4 provides a schematic of an accumulated dose of lidocaine at finite dose.

FIG. 5 provides a schematic of an accumulated dose of lidocaine using various lidocaine base formulations and Lidogel™ as a control formulation at finite dose.

FIG. 6 provides a schematic of an accumulated dose of lidocaine at finite dose.

FIG. 7 provides a schematic of an accumulated dose of lidocaine at finite dose.

FIG. 8 provides a schematic of an accumulated dose of lidocaine at finite dose.

FIG. 9 provides a schematic of an accumulated dose of lidocaine at finite dose.

FIG. 10 provides a schematic of an accumulated dose of lidocaine using various lidocaine HCl formulations at finite dose.

FIG. 11 provides a schematic of an accumulated dose of lidocaine using various lidocaine HCl formulations at finite dose.

FIG. 12 provides a schematic of an accumulated dose of lidocaine using various lidocaine HCl formulations with AMP as a molecular penetration enhancer and no ethanol at finite dose.

FIG. 13 provides a schematic of an accumulated dose of lidocaine using various lidocaine HCl formulations with AMP and other molecular penetration enhancers at finite dose.

FIG. 14 provides a schematic of an accumulated dose of lidocaine using various lidocaine HCl formulations with AMP and lower PG concentrations at finite dose.

FIG. 15 provides a schematic of an accumulated dose of lidocaine using various lidocaine HCl formulations with thickening agents at finite dose.

FIG. 16 provides a schematic of an accumulated dose of lidocaine using various lidocaine HCl formulations with AMP at finite dose.

FIG. 17 provides a schematic of an accumulated dose of lidocaine using various lidocaine HCl formulations with AMP and lower ethanol and PG concentrations at finite dose.

FIG. 18 provides a schematic of an accumulated dose of lidocaine using various lidocaine HCl formulations with AMP in a low solvent vehicle at finite dose.

FIG. 19 provides a schematic of an accumulated dose of lidocaine using various lidocaine HCl formulations at adjusted pH at finite dose.

FIG. 20 provides a schematic of an accumulated dose of lidocaine using various lidocaine HCl formulations at finite dose.

FIG. 21 provides a schematic of an accumulated dose of lidocaine using various lidocaine HCl formulations with no ethanol at finite dose.

FIG. 22 provides a schematic of an accumulated dose of lidocaine using various lidocaine HCl formulations with molecular penetration enhancers at finite dose using shed snake skin.

FIG. 23 provides a schematic of an accumulated dose of lidocaine using various lidocaine HCl formulations at finite dose using cadaver skin.

### DETAILED DESCRIPTION OF THE INVENTION

### I. Definitions

The term “about” as used herein, includes a close, but imprecise quantity of a value. For example, in certain instances the term about includes 5%-10% higher, or 5-10% lower than the value given. For example, “about 10” includes the range of values from 9.5 to 10.5.

The term “transdermal” is used herein to include a process that occurs through the skin. The terms “transdermal” and “percutaneous” are used interchangeably throughout this specification.

The term “topical formulation” is used herein to generally include a formulation that can be applied to skin or a mucosa. Topical formulations may, for example, be used to confer therapeutic benefit to a patient or cosmetic benefits to a consumer. Topical formulations can be used for both topical and transdermal administration of substances.

The term “topical administration” is used herein to generally include the delivery of a substance, such as a therapeutically active agent, to the skin or a localized region of the body.

The term “transdermal administration” is used herein to generally include administration through the skin. Transdermal administration is often applied where systemic delivery of an active is desired, although it may also be useful for delivering an active to tissues underlying the skin with minimal systemic absorption.

The term “molecular penetration enhancer” is used herein to generally include an agent that improves the transport of molecules such as an active agent (e.g., a medicine) into or through the skin. Various conditions may occur at different sites in the body either in the skin or below the skin creating a need to target delivery of compounds. For example, in a treatment for osteoarthritis, the delivery of the active agent into relatively deep underlying joint tissue may be necessary to achieve therapeutic benefit. Thus, a “molecular penetration enhancer” or “MPE” may be used to assist in the delivery of an active agent directly to the skin or underlying tissue or indirectly to the site of the disease through systemic distribution. A molecular penetration enhancer may be a pure substance or may comprise a mixture of different chemical entities.

The term “finite dosing” is used herein to generally include an application of a limited reservoir of a formulation containing an active agent. The reservoir of the active agent is depleted with time leading to a tapering off of the active absorption rate after a maximum absorption rate is reached.

The term “infinite dosing” is used herein to generally include an application of a large reservoir of a formulation containing an active agent. The reservoir is not significantly depleted with time, at least over the time frame intended for the reservoir to be in contact with the skin, thereby providing a long term, continuous steady state of active absorption.

The term “spray-pumpable” is used herein to include formulations, that are liquid at 20° C. under normal atmospheric pressure, that may be dispensed as a spray from a hand-held spray pump dispenser by spraying using normal finger pressure on the portion of the spray pump assembly designed to be activated by finger pressure. By “spray” is meant a jet of finely divided liquid composition. (See, e.g., U.S. Pat. Nos. 3,159,316, 4,034,900, and 4,050,860, which show different spray pump dispensers.) The hand-held spray pump dispenser used to dispense (spray) a composition of this invention typically contains the composition at atmospheric pressure and it is only when finger pressure is applied that the spray pump mechanism temporarily pressurizes the composition to cause a portion of it to leave the dispenser as a spray. The pressure in the mechanism soon returns to atmospheric after the small portion of composition has been dispensed. Such a hand-held spray pump dispenser is considered to be a non-pressurized dispenser. In other words, a feature of this invention is that a hand-held spray pump dispenser (i.e., a non-pressurized dispenser) can be used in its normal manner to dispense the composition of this invention.

The phrase “substantially free” of a lower alcohol is used herein to include “essentially free” of a lower alkanol. Such embodiments may include trace amounts or de minimus amounts of a lower alkanol.

The term “non-stinging,” includes compositions that are substantially without the perception of stinging, pain, or of a distinct discomfort to the user when applied. A stinging test can be used to assess whether the novel topical formulations described herein produce a sensory perception of stinging.

The term “non-irritating,” includes compositions that are substantially non-inflammatory when applied.

### II. Formulations

The present invention provides an aqueous pharmaceutical composition for the management of pain associated with an acute herpes zoster infection. In certain aspects, the composition comprises the following constituents:
a) a topically acting anesthetic active ingredient in a subanesthetic amount;
b) 0% to 5% w/w of a lower alkanol;
c) a molecular penetration enhancer; and
d) a carrier.

In one aspect, the topically acting anesthetic active ingredient includes, but is not limited to, tetracaine, lidocaine, prilocalne, benzocaine, bupivacaine, mepivacaine, dibucaine, etidocaine, butacaine, cyclomethycaine, hexylcaine, proparacaine, lopivacaine and pharmaceutically acceptable salts thereof. In certain preferred aspects, the active ingredient is lidocaine hydrochloride or lidocaine base.

In certain aspects, the amount of topically acting anesthetic active is effective to achieve analgesia without anesthesia i.e., a subanesthetic effective amount. The dose maintains an effective amount of, for example, lidocaine intradermally, for an extended period of time to maintain extended relief from pain. In certain aspects, the topically acting anesthetic active ingredient is in amount of about 1% to about 20% weight by weight (“w/w”). In another embodiment, the topically acting anesthetic active ingredient is in an amount of about 10% to about 20% w/w. In another embodiment, the amount is about 1% to about 10% w/w such as 1, 2, 3, 4, 5, 6, 7, 8, 9, or 10% w/w, and all fractions in between. In other aspects, the amount of topically acting anesthetic active is about 5% to about 10% w/w.

In certain embodiments, the inventive compositions of the present invention are substantially free or essentially free of a lower alkanol. Such embodiments may include trace amounts of a lower alkanol. In other aspects, the composition includes a lower alkanol, such as methanol, ethanol, propanol, isopropanol, butanol, isobutanol and the like or mixtures thereof. In certain embodiments, the alkanol is a C₁-C₄ alkanol, C₂-C₃ alkanol or is ethanol. Preferably, the lower alkanol is used at about 0-5% w/w, such as up to 5% w/w, for example, 0, 1, 2, 3, 4, or 5% w/w, and all fractions in between. In another embodiment, if present, the lower alkanol is used at an amount of up to 3% w/w.

In certain embodiments, the inventive compositions of the present invention includes a molecular penetration enhancer. In certain aspects, the molecular penetration enhancer is a combination of molecular penetration enhancers. In one preferred aspect, the molecular penetration enhancer is a polyhydric alcohol. Such polyhydric alcohols include ethylene glycol, propylene glycol, diethylene glycol, pentamethylene glycol, trimethylene glycol, and the like, or a combination thereof. In one embodiment, the molecular penetration enhancer is propylene glycol. In certain aspects, the molecular penetration enhancer is present in an amount of about 10% to about 50% w/w. In certain preferred aspects, the molecular penetration enhancer is present in an amount of about 10% to about 20% w/w, or about 15% to about 20% such as 15, 16, 17, 18, 19, or 20% w/w, and all fractions in between. The molecular penetration enhancer is preferably non-stinging and non-irritating. In certain aspects, the composition employs a molecular penetration enhancer that allows for transport of the active ingredient (e.g., lidocaine) across the epidermal layer into the dermal layer, while maintaining an effective concentration of the lidocaine in the dermal layer sufficient to relieve pain.

The formulation may also include additional molecular penetration enhancers such as polysorbate 20, methyl laurate, isopropyl palmitate, N-methyl-2-pyrrolidone, aminomethylpropanol (“AMP”), 1,2,6-hexanetriol, methyl salicylate, myristyl lactate, sodium lauryl sulfoacetate or a combination thereof. In one embodiment, the additional molecular penetration enhancer is present in the formulation at about 1% to 10% w/w. In a specific embodiment, the formulation includes 5% to 10% w/w of polysorbate 20 as the additional molecular penetration enhancer.

In certain other embodiments, the inventive compositions of the present invention include a carrier. A preferred carrier is a low-molecular weight PEG. Suitable low-molecular weight PEGs include, but are not limited to, PEG 200, PEG 300, PEG 400, PEG 540, PEG 600, PEG 800, PEG 900, PEG 1000, PEG 1450, PEG 1540 and a combination thereof. In a preferred aspect, the low-molecular weight PEG is PEG 300.

In certain aspects, the carrier is present in an amount up to about 20%. In another embodiment, the carrier is present in an amount up to about 10% w/w, such as 1, 2, 3, 4, 5, 6, 7, 8, 9, or 10% w/w, and all fractions in between.

In certain embodiments, the inventive compositions of the present invention include water. In certain embodiments, the inventive compositions include a water component of more than about 40%, or more than about 50%, such as 60%, 70%, 80% or 90%. In certain instances, the amount of water is about 40% to about 70%, such as 45%, 50%, 55%, 60%, 65%, 70% and all numbers in-between. Water amounts such as 48%, 49%, 50% 51%, 52%, 53%, 54%, 55%, 56%, 57%, 58%, 59%, 60% 61%, 62%, 63%, 64%, 65%, 66%, 67%, 68%, 69% or 70% can be used. In an alternative embodiment, the water is added quantum sufficiat (qs) or as much as suffices.

In certain embodiments, the inventive compositions comprise:
lidocaine base in an amount of about 1% to about 20% w/w;
0% to about 5% w/w of ethanol;
propylene glycol in an amount of about 10% to about 50% w/w; and
PEG-300 in an amount of up to about 20% w/w.

In certain embodiments, the inventive compositions comprise:
lidocaine hydrochloride salt in an amount of about 5% to about 20% w/w;
propylene glycol in an amount of about 16% w/w; and
PEG-300 in an amount of up to about 10% w/w.

In addition, the topical formulations of the present invention can also comprise a pH adjusting agent. In one particular embodiment, the pH adjusting agent is a base. Suitable pH adjusting bases include bicarbonates, carbonates, and hydroxides such as alkali or alkaline earth metal hydroxide as well as transition metal hydroxides. Alternatively, the pH adjusting agent can also be an acid, an acid salt, or mixtures thereof. Further, the pH adjusting agent can also be a buffer. Suitable buffers include citrate/citric acid buffers, acetate/acetic acid buffers, phosphate/phosphoric acid buffers, formate/formic acid buffers, propionate/propionic acid buffers, lactate/lactic acid buffers, carbonate/carbonic acid buffers, ammonium/ammonia buffers, and the like. The pH adjusting agent is preferably sodium hydroxide and is present in an amount sufficient to adjust the pH of the composition to between about pH 4.0 to about 8.5, more preferably about pH 5.5 to about 7.0, such as 6.0 or 6.5.

The present composition may optionally include one or more of the following: glycerine, at least one antioxidant, one chelating agent or a preservative.

The composition can contain 0.001-8%, preferably 0.01-6%, more preferably 0.05-5%, such as 0.06, 0.07, 0.08, 0.09, 0.1, 0.2, 0.3, 0.4, 0.5, 0.6, 0.7, 0.8, 0.9, 1.0, 2.0, 3.0, 4.0, 5.0 (and all fractions in between), by weight of the total composition of a preservative or a combination. A variety of preservatives are suitable, including, but not limited to, benzoic acid, benzyl alcohol, benzylhemiformal, benzylparaben, 5-bromo-5-nitro-1,3-diox-ane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, and DMDM hydantoin. The formulations herein may optionally be sterilized.

Preferred antioxidants for use in the present invention may be selected from the group consisting of butylated hydroxytoluene (“BHT”), butylated hydroxyanisole (“BHA”), ascorbyl linoleate, ascorbyl dipalmitate, ascorbyl tocopherol maleate, calcium ascorbate, carotenoids, kojic acid, tocopherol, tocopherol acetate, tocophereth-5, tocophereth-12, tocophereth-18, tocophereth-80, and mixtures thereof.

Preferred chelating agents may be selected from the group consisting of ethylenediamine tetraacetic acid (“EDTA”), diammonium EDTA, dipotassium EDTA, calcium disodium EDTA, hydroxyethylethylenediaminetriacetic acid (“HEDTA”), ethylenediaminetetraacetic acid, mono(triethanolamine) salt (“TEA-EDTA”), tetrasodium EDTA, tripotassium EDTA, trisodium phosphate, diammonium citrate, galactaric acid, galacturonic acid, gluconic acid, glucuronic acid, humic acid, cyclodextrin, potassium citrate, the potassium salt of ethylenediamine-tetra(methylene phosphonic acid) (“EDTMP”), sodium citrate, sodium EDTMP, and mixtures thereof.

In one embodiment, the formulation is spray-pumpable. For instance, the formulation may be spray-pumpable into a stream of ballistic droplets or a mist to cover the area of treatment. Ideally, the size of the individual droplets produced is large enough so that there is no or very low risk that they are deposited into the respiratory tract. In one example, the droplet size is larger than 5 to 30 microns or 1 to 5 microns. The size of the droplets can be adjusted to ensure optimal delivery of the formulation to the area of need and optimal safety. For example, parameters of the formulation, such as viscosity, or parameters of the delivery device, such as nozzle shape and size and flow rate, can be adjusted as required.

In certain instances, one factor that determines the spray-pumpability of the formulation is viscosity. Viscosity is also a factor that determines how well the formulation sticks to, or does not run off the skin when applied. In a specific example, the viscosity of the formulation is less than 1000 centipose at 20° C. In another example, the viscosity of the formulation is less than 500 centipose at 20° C. In a further example, the viscosity of the formulation is less than 200 centipose at 20° C. In still an additional example, the viscosity of the formulation is less than 100 centipose at 20° C. The viscosity of the formulation can be optimized using pharmaceutically acceptable thickening agents that do not significantly interact with the components of the formulation, do not significantly reduce flux of the formulation or cause stinging or irritation. In one example, one or more of the following thickening agents is used: polyacrylic acid polymers, carbomers, cellulose derivatives, poloxamers, poloxamines dextrans, pectins, natural gums. In one embodiment, cellulose, hydroxyethyl cellulose, hydroxypropyl methyl cellulose, carboxymethyl cellulose or mixtures thereof are used as a thickening agent.

Transdermal Flux

As shown below in the Examples, the present invention provides formulations that display effective rates of transdermal flux. Accordingly, in one embodiment, the present formulation comprises an amount of topically acting anesthetic active suitable to achieve analgesia without anesthesia and having a flux, as determined by a finite dose Franz cell procedure, equal to or greater than the flux of a comparative patch formulation. In one embodiment, the “comparative patch formulation” is the Lidoderm™ patch. In a specific embodiment, the flux is about equal to the flux of the comparative patch formulation. In an alternative embodiment, the flux is greater than the flux of the comparative patch formulation. For example, the flux is 1.2, 1.5, 1.8, 2, 2.5, or 3 times greater than the flux of the comparative patch formulation. In one embodiment, the lag time of delivery of the active through the skin is shorter with the formulations described herein as compared to the comparative patch formulation. In a specific embodiment, the lag time is half that of the comparative patch formulation, which leads to noticeably higher delivery rates within the first hours of application.

### III. Methods of Use

In certain aspects, the compositions and formulations of the invention are particularly suited for use in treating pain associated or resulting from an acute herpes-zoster infection. In certain preferred aspects, the methods employ an anesthetic active agent in an effective amount to achieve analgesia without anesthesia. The formulation is applied to the site of pain typically once, twice, three or four times or as needed per day.

Various modes of application of the inventive formulations can be employed to ensure that a level of an anesthetic active agent is maintained for a time sufficient to substantially reduce the pain accompanying AHZ during the application and frequently after the application has been terminated. The pain accompanying AHZ can be throbbing, stabbing, burning, or lancinating in character and has been shown to be moderate to severe in intensity within 72 hours of rash onset.

In certain instances, the present formulations are spray-on formulations (which may include a propellant) or spray-pumpable formulations, which provides advantages over currently available patch formulations. The formulations of present invention are easier to apply, cover a larger surface area, are non-stinging and can be applied without touching the skin surface with other than the formulation itself. The skin surfaces to which the formulations of the current invention can be applied include, but are not limited to, skin such as the chest region (thoracic), the forehead (trigeminal) or wherever the herpes rash occurs. In addition, the formulations can be applied to other surfaces such as mucosal surfaces, genitals, anus, nail surface, wound surface, rash surface, bed sore surface, and diabetes-induced ulcerous skin surface.

In other aspects, the compositions and formulations of the invention are particularly suited for use in treating pain associated with postherpetic neuralgia. The invention provides a method for administering a local anesthetic agent to a patient to treat or prevent pain. The method involves topically administering a pharmaceutical composition as described herein to treat patients suffering from pain associated with a skin condition or disorder, e.g., an insect bite, muscle pain, arthritis, allergic reaction, rash (e.g., a rash caused by poison oak or poison ivy), itch, blister, sore nail, corn, mechanical puncture (e.g., catheterization and needle injection), laser treatment, or any combination thereof.

In certain embodiments, the present composition is a foam or foamable. The composition herein can be placed in an aerosol (e.g., pressurized) container and combined with a liquefied gas propellant, the composition being stable in its predispensed state. Typically, from about 2% to about 18% w/w of an aerosol propellant is used, preferably 3% to about 7% is used, wherein the other ingredients are used proportionally. Generally, the aerosol propellant is selected from a hydrocarbon, a chlorofluorocarbon, or a mixture thereof.

In certain other aspects, the foam embodiment optionally includes a hydrophobic solvent such as a vegetable oil (e.g., corn or soybean oil). In another embodiment, the composition is foamable without the need of a liquefied gas propellant. In one aspect, the foamable embodiment optionally includes a surfactant at up to 10% w/w. In one example, the surfactant is polysorbate 20.

In still yet another aspect, the composition is selected from the group of a gel, a cream, an emulsion, a lotion, an organogel, an ointment, and a solution. More preferably, the composition is a solution.

The method may also be used to treat patients suffering from breakthrough pain, migraine, neuropathic pain, and angina pain. In addition, the compositions and systems of the invention may be administered with a wound dressing to treat burns, wounds and scrapes.

Advantageously, the compositions and drug delivery systems described herein can also be used as part of a pre-treatment regimen used to prevent or minimize the pain associated with other topical therapies, medical procedures or cosmetic procedures.

### IV. Examples

Table 1 provides a list of the materials used in the experiments described herein and a list of the abbreviations used for the chemical compounds.

TABLE 1

| Abbr | Chemical | CAS |
|---|---|---|
| Acetone | Acetone | 67-64-1 |
| ALS | Ammonium lauryl sulfate | 2235-54-3 |
| AMP | 2-Amino-2-methyl-1-propanol | 124-68-5 |
| BenzOH | Benzyl Alcohol | 100-51-6 |
| ButGly | Butylene glycol | 107-88-0 |
| C70 | Sodium lauryl ether(2) sulfate | 3088-31-1 |
| CB | Chlorobutanol | 6001-64-5 |
| CitrAC | Citric Acid | 77-92-9 |
| DimIso | Dimethyl Isosorbide | 5306-85-4 |
| DioPht | Dioctyl Phthalate | 117-81-7 |
| DMSO | Dimethyl Sulfoxide | 67-68-5 |
| EO | Ethyl Oleate | 111-62-6 |
| EthAce | Ethyl Acetate | 141-78-6 |
| EtOH | Ethanol | 64-17-5 |
| GL | Glyceryl Laurate (glycerol | 142-18-7 |
| | monolaurate) | |
| Gly | Glycerine | 56-81-5 |
| GO | Glyceryl Oleate (glycerol | 111-03-5 |
| | monooleate) | |
| GR | Glyceryl Ricinoleate | 68459-67-6 |
| HexGly | Hexylene glycol | 107-41-5 |
| HexTri | 1,2,6 Hexanetriol | 106-69-4 |
| IM | Isopropyl Myristate | 110-27-0 |
| IPA | Isopropyl Alcohol | 67-63-0 |
| IsoPal | Isopropyl Alcohol | 142-91-6 |
| Lactic | Lactic Acid | 50-21-5 |
| Lidocaine | Lidocaine base | 137-58-6 |
| Lidocaine HCL | Lidocaine hydrochloride | 6108-05-0 |
| LL | Lauryl Lactate | 6283-92-7 |
| MetPar | Methyl Paraben | 99-76-3 |
| ML | Methyl Laurate | 111-82-0 |
| MP | 1-methyl-2-Pyrrolidone | 872-50-4 |
| MS | Methyl Salicylate | 119-36-8 |
| OD | Octyl Dodecanol | 5333-42-6 |
| Oleic | Oleic Acid (octadecenoic acid) | 112-80-1 |
| OleylOH | Oleyl Alcohol | 143-28-2 |
| Peg300 | Poly(ethylene glycol) 300 | 25322-68-3 |
| Peg400 | Poly(ethylene glycol) 400 | 9004-81-3 |
| PG | Propylene Glycol | 57-55-6 |
| PGDCap | Propylene Glycol Dicaprylate | 68583-51-7 |
| PropPar | Propyl Paraben | 94-13-3 |
| SLSA | Sodium Lauryl Sulfoacetate | 1847-58-1 |
| Sp20 | Span 20 (sorbitan monolaurate) | 1338-39-2 |
| Terp | alpha-terpineol | 98-55-5 |
| TLS | Triethanolamine Lauryl Sulfate | 139-96-8 |
| Tw20 | Tween 20 (POE sorbitan | 9005-64-5 |
| | monolaurate) | |
| Tw60 | Tween 60 (POE sorbitan | 9005-67-8 |
| | monolaurate) | |
| Tw80 | Tween 80 (POE sorbitan | 9005-65-6 |
| | monolaurate) | |

### Example 1

### a) Methods

Franz diffusion cell (FDC) experiments were used to analyze lidocaine base and lidocaine hydrochloride flux rates from varying formulations across a substrate membrane.

Franz diffusion cells are a common and well known method for measuring transdermal flux rates. The general Franz cell procedure is described in Franz, T. J., Percutaneous absorption: on the relevance of in vitro data: J. Invest Derin, 64:190-195 (1975). The following was the methodology used in the present Examples.

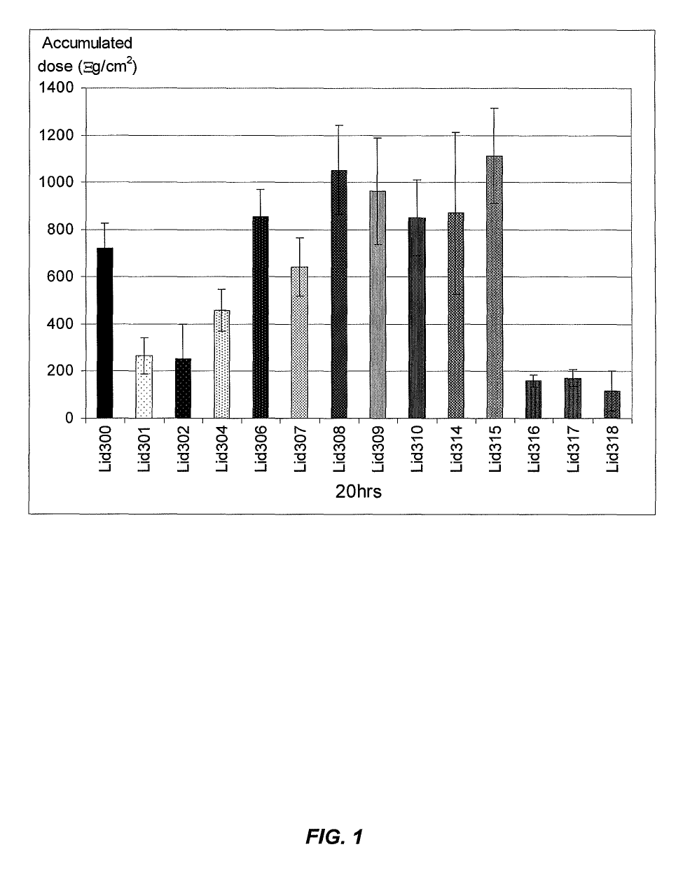

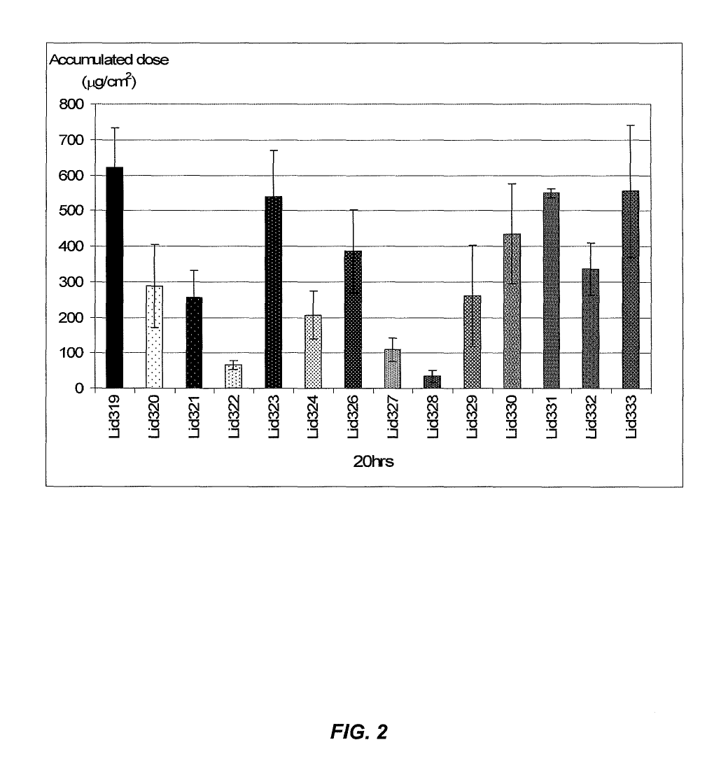

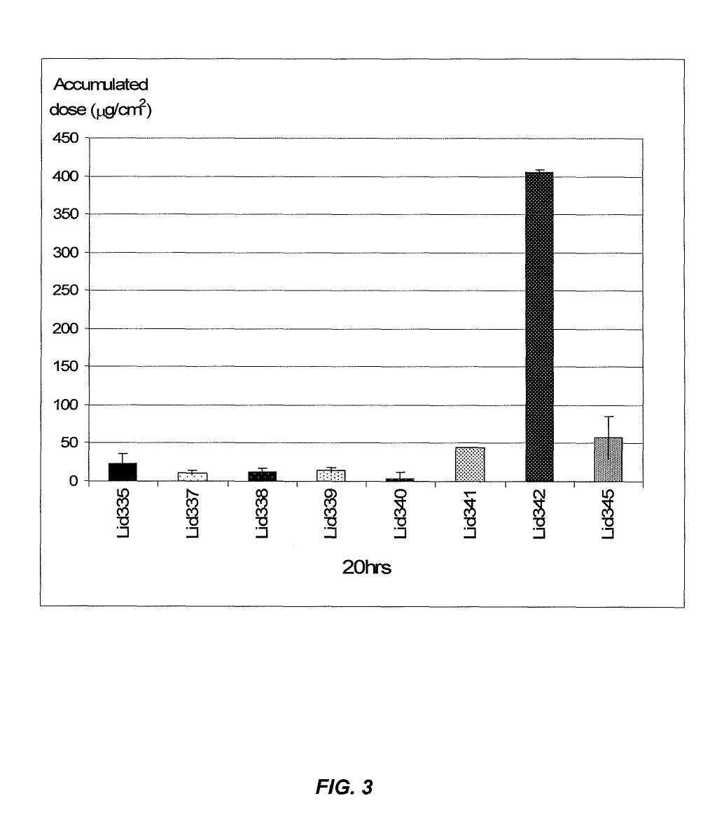

Franz cells with a 3 ml receptor well volume were used in conjunction with split thickness cadaver skin (0.015″-0.018″, AlloSource), dermatomed porcine skin (Lampire Biologicals), or shed snake skin (Python regius). Porcine skin was used in the experiments depicted in FIGS. 1 and 3-9, snake skin was used in the experiments depicted in FIG. 2 and cadaver skin was used in the remaining experiments described herein. The donor well had an area of ˜0.5 cm². Receptor wells were filled with isotonic phosphate buffered saline (PBS) doped with 0.01% sodium azide. The flanges of the Franz cells were coated with vacuum grease to ensure a complete seal and were clamped together with uniform pressure using a pinch clamp (SS #18 VWR 80073-350). After Franz cells were assembled, the skin was allowed to pre-hydrate for ˜45 minutes. Dosing levels varied from 2 mg/cm² (considered finite dose) to 200 mg/cm² (considered infinite dose). The Franz cells were maintained at 32° C. by placement in a humidified incubator. The receptor wells of the Franz cells were agitated at all times with a stir bar. Sample aliquots were drawn from the receptor wells at varying time points and replaced with fresh buffer. Measurements for each formulation were carried out in six-fold replicates. The concentration of the active in the sample aliquots was analyzed using high performance liquid chromatography. In certain experiments, Lidoderm™ patch was used as a control. For experiments wherein the retention of lidocaine was measured in the skin, the skin was collected, washed of excess formulation on the stratum corneum, then homogenized in a ethanol solution. After a period of one day, the lidocaine was extracted from the skin into the ethanol solution. An aliquot of the ethanol was then taken and measured for lidocaine concentration.

### b) Formulation Compositions

Table 2 provides a list of formulations which are used herein. All values listed are in wt %.

TABLE 2
Examples of formulations
Components in wt/wt %

| Formula- | | | | | Ace- | | Citr- | Hex- | Iso- | | Hex- | Dim- | Lido- |
|---|---|---|---|---|---|---|---|---|---|---|---|---|---|
| tion | PG | Gly | EtOH | Water | tone | ALS | AC | Gly | Pal | TLS | Tri | iso | caine |
| Lid300 | 39 | 2 | 15 | 39 | 10 | | | | | | | | 5 |
| Lid301 | 39 | 2 | 15 | 39 | | 5 | | | | | | | 5 |
| Lid302 | 39 | 2 | 15 | 39 | | | 10 | | | | | | 5 |
| Lid304 | 39 | 2 | 15 | 39 | | | | 12 | | | | | 5 |
| Lid306 | 39 | 2 | 15 | 39 | | | | | 6 | | | | 5 |
| Lid307 | 39 | 2 | 15 | 39 | | | | | | 10 | | | 5 |
| Lid308 | 39 | 2 | 15 | 39 | | | | | | | 8 | | 5 |
| Lid309 | 39 | 2 | 15 | 39 | | | | | | | | 15 | 5 |
| Formula- | | | | | | Eth- | | | | Lido- | | | |
| tion | PG | Gly | EtOH | Water | Sp20 | Ace | Terp | XSNa | Urea | caine | | | |
| Lid310 | 39 | 2 | 15 | 39 | | | | | | 5 | | | |
| Lid314 | 39 | 2 | 15 | 39 | 10 | | | | | 5 | | | |
| Lid315 | 39 | 2 | 15 | 39 | | 15 | | | | 5 | | | |
| Lid316 | 39 | 2 | 15 | 39 | | | 5 | | | 5 | | | |
| Lid317 | 39 | 2 | 15 | 39 | | | | 10 | | 5 | | | |
| Lid318 | 39 | 2 | 15 | 39 | | | | | 10 | 5 | | | |
| Formula- | | | | | | | | Oleyl- | | | | | Lido- |
| tion | PG | Gly | EtOH | Water | MP | AMP | Oleic | OH | MS | GR | ML | OD | caine |
| Lid319 | 39 | 2 | 15 | 39 | 10 | | | | | | | | 5 |
| Lid320 | 39 | 2 | 15 | 39 | | 1 | | | | | | | 5 |
| Lid321 | 39 | 2 | 15 | 39 | | | 5 | | | | | | 5 |
| Lid322 | 39 | 2 | 15 | 39 | | | | 5 | | | | | 5 |
| Lid323 | 39 | 2 | 15 | 39 | | | | | 1 | | | | 5 |
| Lid324 | 39 | 2 | 15 | 39 | | | | | | 2 | | | 5 |
| Lid326 | 39 | 2 | 15 | 39 | | | | | | | 1 | | 5 |
| Lid327 | 39 | 2 | 15 | 39 | | | | | | | | 8 | 5 |
| Formula- | | | | | | | Dio- | | Myr- | | Lido- | | |
| tion | PG | Gly | EtOH | Water | Lactic | Tw20 | Pht | EO | Lac | W143 | caine | | |
| Lid328 | 39 | 2 | 15 | 39 | 15 | | | | | | 5 | | |
| Lid329 | 39 | 2 | 15 | 39 | | 20 | | | | | 5 | | |
| Lid330 | 39 | 2 | 15 | 39 | | | 15 | | | | 5 | | |
| Lid331 | 39 | 2 | 15 | 39 | | | | 2 | | | 5 | | |
| Lid332 | 39 | 2 | 15 | 39 | | | | | 4 | | 5 | | |
| Lid333 | 39 | 2 | 15 | 39 | | | | | | 2 | 5 | | |
| Formula- | | | | | | | | | | | | | Lido- |
| tion | PG | Gly | EtOH | Water | SNLS | Peg300 | Gly | MS | SLSA | Tbutyl | Lim | IPA | caine |
| LidBase | 39 | 2 | 15 | 39 | | | | | | | | | 5 |
| Lid335 | 39 | 2 | 15 | 39 | 6 | | | | | | | | 5 |
| Lid337 | 39 | 2 | 15 | 39 | | 25 | | | | | | | 5 |
| Lid338 | 39 | 2 | 15 | 39 | | | 20 | | | | | | 5 |
| Lid339 | 39 | 2 | 15 | 39 | | | | 1 | | | | | 5 |
| Lid340 | 39 | 2 | 15 | 39 | | | | | 6 | | | | 5 |
| Lid341 | 39 | 2 | 15 | 39 | | | | | | 1 | | | 5 |
| Lid342 | 39 | 2 | 15 | 39 | | | | | | | 2 | | 5 |
| Lid345 | 39 | 2 | 15 | 39 | | | | | | | | 20 | 5 |
| Formula- | | | | | Hex- | | | | Lido- | | | | |
| tion | PG | Gly | EtOH | Water | Tri | Sp20 | MS | Lim | caine | | | | |
| Lidoderm | | | | | | | | | | | | | |
| Lid308b | 35 | 2 | 15 | 35 | 8 | | | | 5 | | | | |
| Lid335b | 36 | 2 | 15 | 36 | | 6 | | | 5 | | | | |
| Lid323b | 38.5 | 2 | 15 | 39 | | | 0.5 | | 5 | | | | |
| Lid342b | 38 | 2 | 15 | 38 | | | | 2 | 5 | | | | |
| Formula- | | | | | Hex- | Eth- | | Lido- | | | | | |
| tion | PG | Gly | EtOH | Water | Tri | Ace | Tw20 | caine | | | | | |
| Lidoderm | | | | | | | | | | | | | |
| Lid308b | 35 | 2 | 15 | 35 | 8 | | | 5 | | | | | |
| Lid308c | 37.5 | 2 | 12 | 37.5 | 6 | | | 5 | | | | | |
| Lid308f | 31 | 2 | 15 | 31 | 6 | 10 | | 5 | | | | | |
| Lidogel | 90 | | | | | | 4 | 5 | | | | | |
| Formula- | | | | | Hex- | | Lido- | | | | | | |
| tion | PG | Gly | EtOH | Water | Tri | MS | caine | | | | | | |
| Lid323b | 38.5 | 2 | 15 | 39 | | 0.5 | 5 | | | | | | |
| Lid354 | 63 | 2 | | 24 | 6 | | 5 | | | | | | |
| Lid355 | 60 | 2 | 5 | 22.5 | | 0.5 | 10 | | | | | | |
| Lid356 | 66 | 2 | | 26.5 | | 0.5 | 5 | | | | | | |
| Formula- | | | | | Hex- | Lido- | | | | | | | |
| tion | PG | Gly | EtOH | Water | Tri | caine | | | | | | | |
| Lid350 | 47 | 2 | 5 | 30 | 6 | 10 | | | | | | | |
| Lid370 | 50 | 2 | 5 | 33 | 0 | 10 | | | | | | | |
| Lid371 | 44 | 2 | 5 | 27 | 12 | 10 | | | | | | | |
| Lid372 | 60 | 2 | 5 | 17 | 6 | 10 | | | | | | | |
| Lid373 | 77 | 2 | 5 | 0 | 6 | 10 | | | | | | | |
| Formula- | | | | | | Lido- | | | | | | | |
| tion | PG | Gly | EtOH | Water | MS | caine | | | | | | | |
| Lid375 | 60 | 2 | 5 | 23 | | 10 | | | | | | | |
| Lid376 | 60 | 2 | 5 | 22 | 1 | 10 | | | | | | | |
| Lid377 | 60 | 2 | 5 | 21 | 2 | 10 | | | | | | | |
| Lid378 | 49 | 2 | 5 | 33 | 1 | 10 | | | | | | | |
| Lid379 | 82 | 2 | 5 | 0 | 1 | 10 | | | | | | | |
| Lid380 | 40 | 2 | 5 | 42 | 1 | 10 | | | | | | | |
| Formula- | | | | | Hex- | Lido- | | | | | | | |
| tion | PG | EtOH | Water | Gly | Tri | caine | | | | | | | |
| Lid500 | 42 | 5 | 38.5 | 2 | 7.5 | 5 | | | | | | | |
| Formula- | | | | | Lido- | Hex- | | | Myr- | | | | |
| tion | PG | EtOH | Water | Gly | caine | Tri | Tw20 | AMP | Lac | MP | LL | Tw20 | |
| Lidoderm | Lidoderm | | | | | | | | | | | | |
| | patch | | | | | | | | | | | | |
| L504 | 45 | 5 | 35.5 | 2 | 5 | 7.5 | | | | | | | |
| L506 | 49 | 5 | 39 | 2 | 5 | | | | | | | | |
| L507 | 48 | 5 | 39 | 2 | 5 | | | 1 | | | | | |
| L508 | 46.25 | 5 | 37 | 2 | 5 | | 6 | | 1 | | | | |
| L509 | 43 | 5 | 35 | 2 | 5 | | | | | 10 | | | |
| Formula- | | | | Lido- | | | Benz- | | | | | | |
| tion | PG | EtOH | Water | HCL | AMP | MP | OH | ML | GL | ALS | NaCS | | |
| Emla | Emla | | | | | | | | | | | | |
| | cream | | | | | | | | | | | | |
| LidHCl1 | 27 | | 63 | 10 | | | | | | | | | |
| LidHCl2 | 27 | 6 | 62 | 10 | 1 | | | | | | | | |
| LidHCl3 | 27 | | 58 | 10 | | 5 | | | | | | | |
| LidHCl4 | 27 | | 60 | 10 | | | 3 | | | | | | |
| LidHCl5 | 27 | | 62 | 10 | | | | 1 | | | | | |
| LidHCl6 | 27 | 12 | 61.5 | 10 | 1 | | | | 0.5 | | | | |
| LidHCl7 | 27 | | 61 | 10 | | | | | | 2 | | | |
| LidHCl9 | 27 | 6 | 61.5 | 10 | | | | | 0.5 | | 1 | | |
| Formula- | | | | Lido- | | | | | | | | | |
| tion | PG | EtOH | Water | HCL | AMP | GL | ALS | TEA | LD | | | | |
| LidHCl20a | 25.5 | 5.5 | 59 | 10 | | | | | | | | | |
| LidHCl8a | 25.5 | 5.5 | 58.3 | 10 | 0.7 | | | | | | | | |
| LidHCl2b | 35.5 | 5.5 | 48.3 | 10 | 0.7 | | | | | | | | |
| LidHCl2c | 35.5 | 5.5 | 48.65 | 10 | | 0.35 | | | | | | | |
| LidHCl2f | 35.5 | 5.5 | 43.5 | 10 | | | 5.5 | | | | | | |
| LidHCl2g | 25.5 | 5.5 | 58.3 | 10 | | | | 1 | | | | | |
| LidHCl2d | 35.5 | 5.5 | 47.6 | 10 | 0.7 | | | | 0.7 | | | | |
| LidHCl2e | 35.5 | 5.5 | 53.65 | 5 | 0.35 | | | | | | | | |
| Formula- | | | | Lido- | | | | | | | | | |
| tion | PG | EtOH | Water | cain HC [symbol] | AMP | C-70 | SLSA | | | | | | |
| Lid25 | 37.5 | | 51.8 | 10 | 0.7 | | | | | | | | |
| Lld26 | 32.5 | | 56.8 | 10 | 0.7 | | | | | | | | |
| Lid28 | 37.5 | | 50.8 | 10 | 0.7 | 1 | | | | | | | |
| Lid29 | 37.5 | | 49.8 | 10 | 0.7 | | 1 | | | | | | |
| Formula- | | | Lido- | | | | Polox- | | | | | | |
| tion | PG | Water | HCL | AMP | LL | Tw20 | 407 | MS | | | | | |
| LidHCl31 | 51.8 | 37.5 | 10 | 0.7 | | | | | | | | | |
| LidHCl32 | 46.8 | 37.5 | 10 | 0.7 | | 5 | | | | | | | |
| LidHCl33 | 51.3 | 37.5 | 10 | 0.7 | | 2.3 | | 0.5 | | | | | |
| LidHCl34 | 47.8 | 35.5 | 10 | 0.7 | 1 | 5 | | | | | | | |
| LidHCl35 | 47.8 | 36.2 | 10 | | 1 | 5 | | | | | | | |
| LidHCl36 | 46.8 | 37.5 | 10 | 0.7 | | | 5 | | | | | | |
| Formula- | | | | Lido- | | | Hex- | | | | | | |
| tion | PG | EtOH | Water | HCL | AMP | Tw20 | Tri | Gly | | | | | |
| LidHCl49 | 36 | 5 | 48.3 | 10 | 0.7 | | | | | | | | |
| LidHCl51 | 33 | 5 | 45.3 | 10 | 0.7 | 4 | | 2 | | | | | |
| Formula- | | | | But- | | Lido- | | | | | | | |
| tion | PG | EtOH | Water | Gly | PEG300 | HCL | AMP | | | | | | |
| LidHCl55 | 25 | 3 | 51.3 | | 10 | 10 | 0.7 | | | | | | |
| LidHCl56 | 20 | 3 | 51.3 | | 15 | 10 | 0.7 | | | | | | |
| LidHCl57 | | 5 | 48.3 | 36 | | 10 | 0.7 | | | | | | |
| LidHCl58 | | 3 | 51.3 | 25 | 10 | 10 | 0.7 | | | | | | |
| Formula- | | | | Lido- | | | | | [symbol] ropyl | | | | |
| tion | PG | EtOH | Water | HCL | AMP | PEG300 | HPMC | HY117 | Paraben | | | | |
| LidHCl49d | 35 | 5 | 46.1 | 10 | 0.7 | | | 3 | 0.2 | | | | |
| LidHCl49e | 35.5 | 5 | 48.3 | 10 | 0.7 | | 0.5 | | | | | | |
| LidHCl55b | 25 | 3 | 48.1 | 10 | 0.7 | 10 | | 3 | 0.2 | | | | |
| LidHCl56b | 20 | 3 | 48.1 | 10 | 0.7 | 15 | | 3 | 0.2 | | | | |
| Formula- | | | | Lido- | | | But- | | [symbol] ropyl | | | | |
| tion | PG | EtOH | Water | HCL | AMP | PEG300 | Gly | HY117 | Paraben | | | | |
| LidHCl49g | 35 | 5 | 48.1 | 10 | 0.7 | | | 1 | 0.2 | | | | |
| LidHCl55 | 25 | 3 | 51.3 | 10 | 0.7 | 10 | | | | | | | |
| lidHCl55c | 25 | 3 | 50.1 | 10 | 0.7 | 10 | | 1 | 0.2 | | | | |
| LldHCl56c | 20 | 3 | 51.1 | 10 | 0.7 | 15 | | | 0.2 | | | | |
| LidHCl58b | | 3 | 51.1 | 10 | 0.7 | 10 | 25 | | 0.2 | | | | |
| Formula- | | | | Lido- | | | Eth- | | | | | | |
| tion | PG | EtOH | Water | HCL | AMP | PEG300 | Ace | | | | | | |
| LidHCl65 | 20 | 3 | 46.3 | 10 | 0.7 | 16 | 5 | | | | | | |
| LidHCl66 | 20 | 3 | 41.3 | 10 | 0.7 | 15 | 10 | | | | | | |
| LidHCl67 | 20 | 3 | 51.3 | 10 | 0.7 | 10 | 5 | | | | | | |
| Formula- | | | | Lido- | | | | | | | | | |
| tion | PG | EtOH | Water | HCL | AMP | PEG300 | | | | | | | |
| LidHCl70 | 36 | 3 | 50.3 | 10 | 0.7 | | | | | | | | |
| LidHCl71 | 30 | 3 | 50.3 | 10 | 0.7 | 6 | | | | | | | |
| LidHCl72 | 15 | 1.5 | 51.3 | 10 | 0.7 | 21.5 | | | | | | | |
| LidHCl73 | 25 | 1.5 | 51.3 | 10 | 0.7 | 11.5 | | | | | | | |
| LidHCl74 | 20 | 1.5 | 51.3 | 10 | 0.5 | 16.7 | | | | | | | |
| Formula- | | | | Lido- | | | ALS | | | [symbol] djusted | | | |
| tion | PG | EtOH | Water | HCL | AMP | PEG300 | (28%) | TLS | SLSA | w/NaOH) | | | |
| LidHCl56.1 | 20 | 3 | 51.4 | 10 | 0.6 | 15 | | | | 7 | | | |
| LidHCl75 | 17 | 1.5 | 46.9 | 20 | 0.6 | 14 | | | | | | | |
| LidHCl76 | 20 | 3 | 52 | 10 | | 15 | | | | 6.93 | | | |
| LidHCl77 | 15 | 1.5 | 63.5 | 10 | | | 10 | | | | | | |
| LidHCl78 | 15 | 1.5 | 60.5 | 10 | | 10 | | 3 | | | | | |
| LidHCl80 | 15 | 1.5 | 61.2 | 10 | 0.3 | 10 | | | 2 | | | | |
| Formula- | | | | Lido- | | | | | | [symbol] djusted | | | |
| tion | PG | EtOH | Water | HCL | AMP | PEG300 | SLSA | LL | Tw20 | w/NaOH) | | | |
| LidHCl100 | 16.4 | 1.5 | 55.2 | 10 | 0.4 | 16.5 | | | | | | | |
| LidHCl101 | 15 | 1.5 | 61.5 | 10 | | 10 | 2 | | | 6.48 | | | |
| LidHCl102 | 15 | 1.5 | 59.5 | 10 | | 10 | 4 | | | 6.48 | | | |
| LidHCl103 | 15 | 1.5 | 61.5 | 10 | | 10 | 2 | | | 5.97 | | | |
| LidHCl104 | 15 | 1.5 | 63.5 | 10 | | 10 | | | | 6.48 | | | |
| LidHCl105 | 16 | 1.5 | 53.5 | 10 | | 10 | | 2 | 8 | 6.44 | | | |
| Formula- | | | | Lido- | | | | | | pH(adjusted | | | |
| tion | PG | EtOH | Water | HCL | PEG300 | SLSA | LL | Tw20 | | w/NaOH) | | | |
| LidHCl106 | 15 | 1.5 | 63.5 | 10 | 10 | | | | | 6.3 | | | |
| LidHCl109 | 16.5 | | 57.5 | 10 | 10 | | 1 | 5 | | 6.02 | | | |
| LidHCl110 | 16.5 | | 51.5 | 10 | 10 | | 2 | 10 | | 6.04 | | | |
| LidHCl111 | 15 | 1.5 | 53.5 | 20 | 10 | | | | | 5.95 | | | |
| LidHCl112 | 16.5 | | 53.5 | 10 | 10 | | | 10 | | 6.04 | | | |
| LidHCl113 | 15 | 1.5 | 51.5 | 20 | 10 | 2 | | | | 6.01 | | | |
| LidHCl114 | 16.5 | | 63.5 | 10 | 10 | | | | | 6.3 | | | |
| Formula- | | | Lido- | | | | [symbol] ropyl | [symbol] ethyl | | pH(adjusted | | | |
| tion | PG | Water | HCL | PEG 300 | SLSA | Tw20 | Parab [symbol] | Paraben | | w/NaOH) | | | |
| LidHCL115a | 16.5 | 63.3 | 10 | 10 | | | 0.1 | 0.1 | | 6 | | | |
| LidHCL116a | 16.5 | 61.3 | 10 | 10 | 2 | | 0.1 | 0.1 | | 6 | | | |
| LidHCL117a | 16.5 | 53.3 | 10 | 10 | | 10 | 0.1 | 0.1 | | 6 | | | |
| LidHCL117b | 16.5 | 57.3 | 10 | 10 | | 6 | 0.1 | 0.1 | | 6 | | | |

[symbol]

indicates data missing or illegible when filed

### c) Results

2. Screening Varying Molecular Penetration Enhancers with Lidocaine Base

Lidocaine base formulations were prepared in a propylene glycol (PG), ethanol (EtOH), and a water rich vehicle and screened with various molecular penetration enhancers. The initial vehicle (Vehicle 1) was set with PG˜40%, EtOH˜15%, and water˜40%. The results from the initial screening are shown in FIGS. 1-3.

The following are preferred formulations:
1. Lid306 (containing IsoPal), Lid309 (containing HexTri), Lid310 (containing BenzOH), Lid315 (containing EthAce);
2. Lid319 (containing MP), Lid323 (containing MS), Lid331 (containing EO), Lid315 (containing W143); and
3. Lid342 (containing Lim).

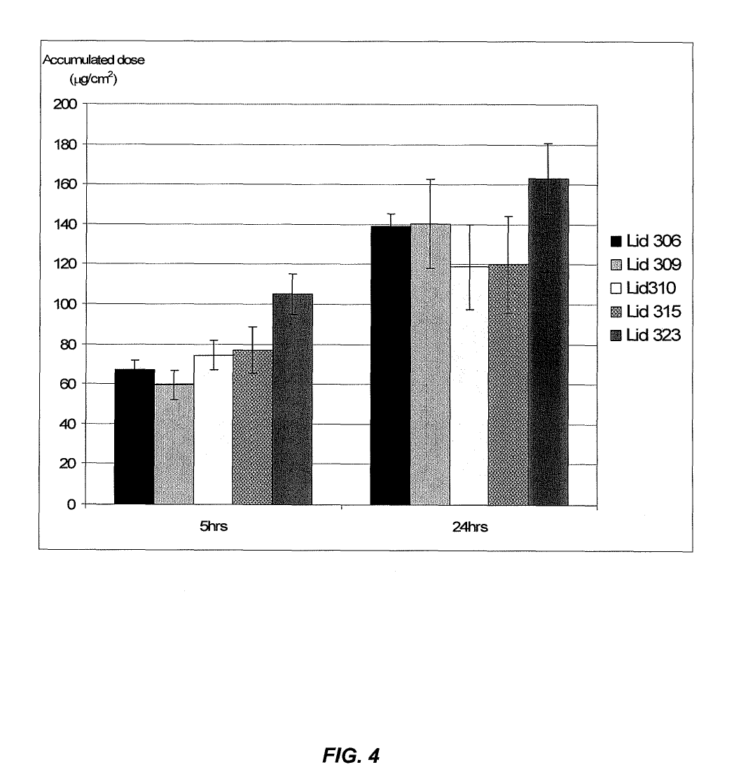

After the initial molecular penetration enhancer screening was complete at infinite dosing, follow up studies were carried out at finite dosing. Formulations that continued to show performance were iteratively varied and tested. FIGS. 4-9 show the results of these follow-up studies.

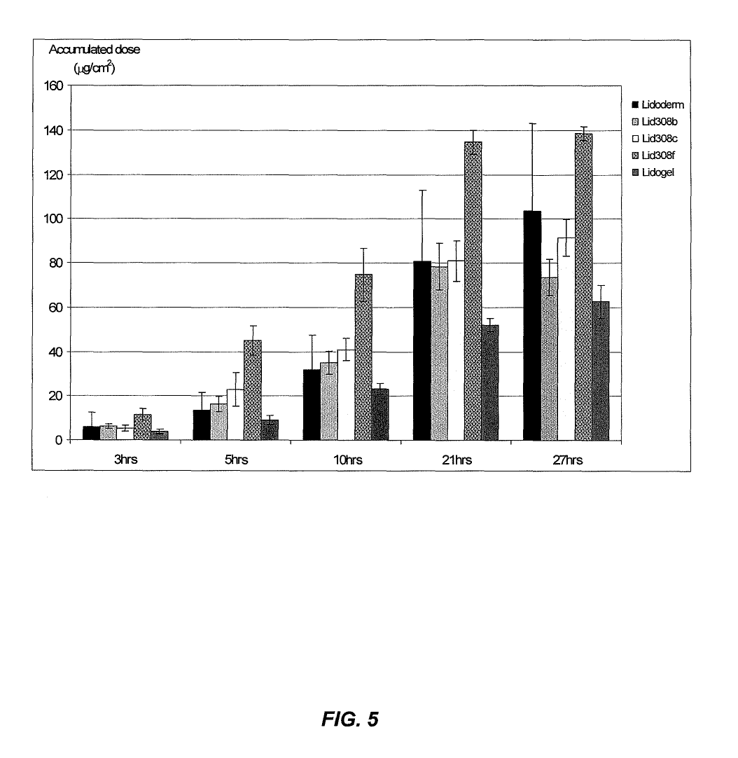

FIG. 4 shows that Lid315 and Lid306 performed well. HexTri (Lid308) and MS (Lid323) were noted as molecular penetration enhancers of interest. FIG. 5 shows the results of various formulations that include HexTri as a molecular penetration enhancer. The addition of EthAce (Lid308f) noticeably increased the flux rate from the base Lid308 formulation. FIG. 5 also shows the flux results of a high PG containing formulation (Lidogel) as compared to Lidoderm and some lower PG containing formulations.

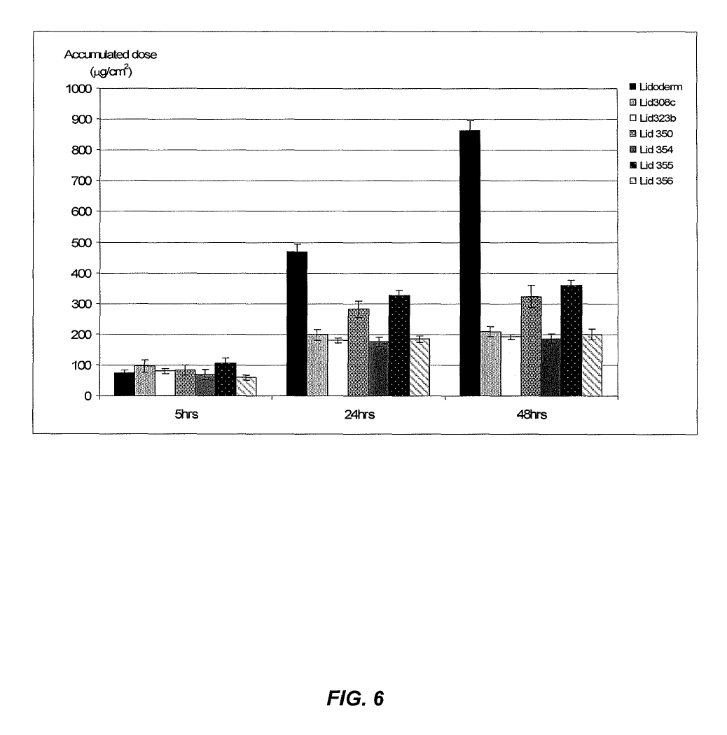

FIG. 6 shows the results of various HexTri and MS containing formulations. These HexTri and MS formulations showed an increase in flux when the PG constituent in vehicle 1 was increased (and the EtOH concentration was decreased to 5%).

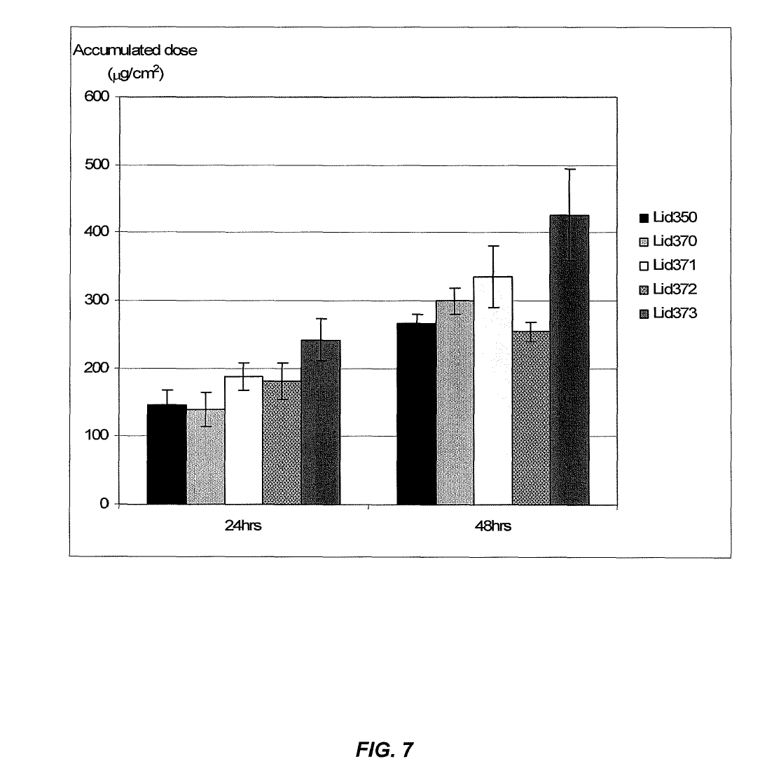

FIG. 7 shows various formulations that include HexTri as a molecular penetration enhancer. The data shows that increasing water concentration leads to a lowered flux (comparing Lid350 to Lid373). In addition, the data shows that increasing the HexTri concentration leads to a corresponding increase in flux (comparing Lid371 to Lid 370).

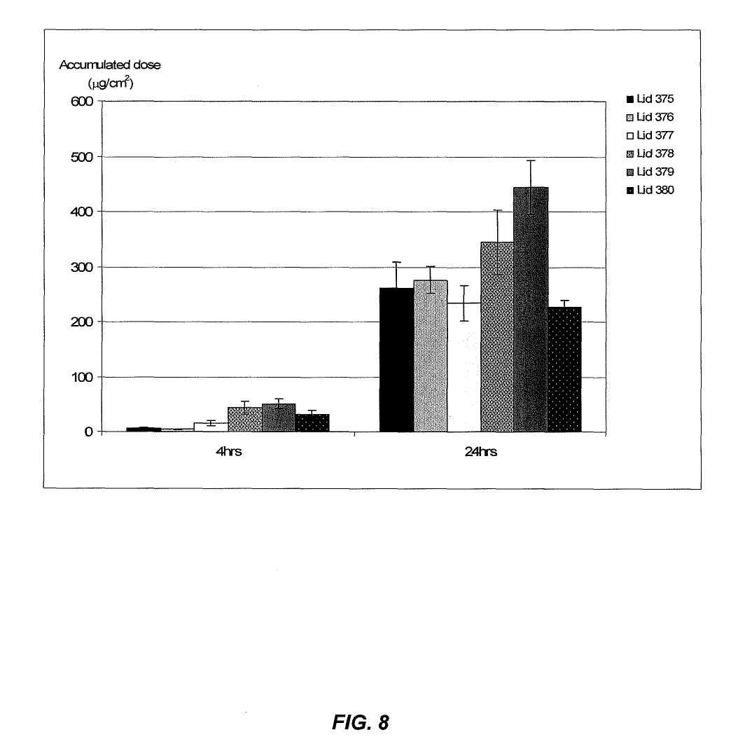

FIG. 8 shows various formulations that include MS as a molecular penetration enhancer. High PG concentrations lead to an increase in flux (Lid379). Lid378 is of particular interest.

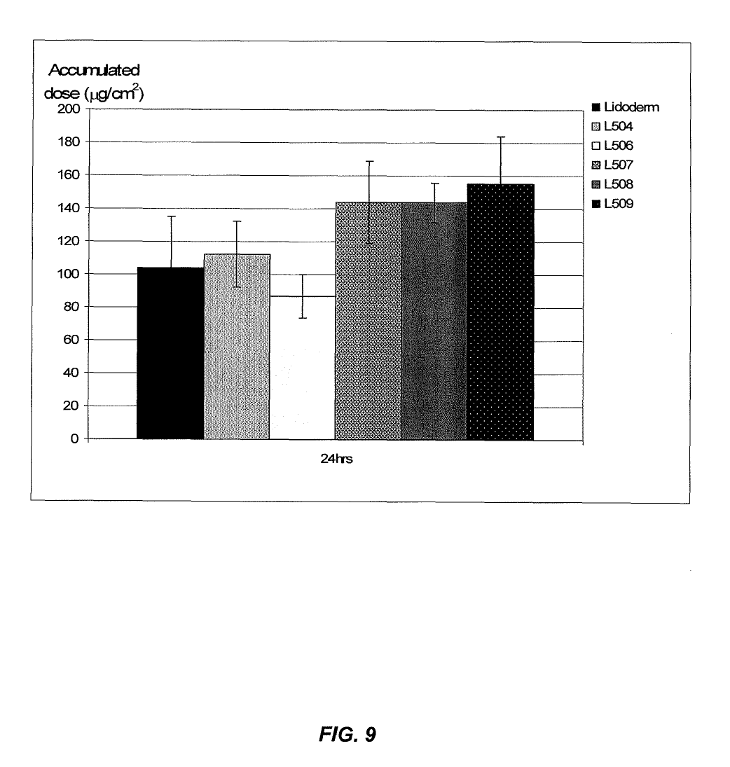

HexTri continued to show performance as a mild molecular penetration enhancer (L504 compared to L506) and was tested in further experiments (see, FIG. 9). In this experiment, the water concentration in L504 was maximized in order to develop a more benign vehicle with reduced chance of irritation. At a water concentration greater than about 40% w/w, the lidocaine tended to crystallized with time.

### Example 2

### Screening Varying Molecular Penetration Enhancers with Lidocaine Hydrochloride

Lidocaine hydrochloride (Lidocaine HCl) was also examined. As lidocaine HCl is more soluble in water than lidocaine base, it was possible to make formulations with a higher water component.

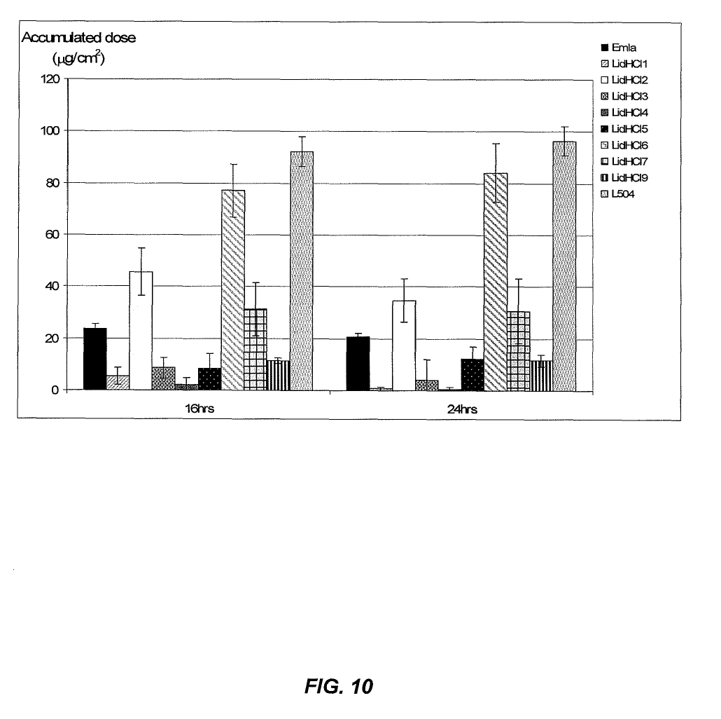

FIGS. 10 to 22 show the results of screening with lidocaine HCl in a water based vehicle with the incorporation of various molecular penetration enhancers. FIG. 10 shows that the flux of formulations using lidocaine HCl was noticeably lower than the L504 comparator (lidocaine base formulation), with the notable exception of LidHCl6. This formulation used AMP and GL as a penetration enhancement combination, leading to flux comparable to L504 with even though the water content was increased by ˜75%.

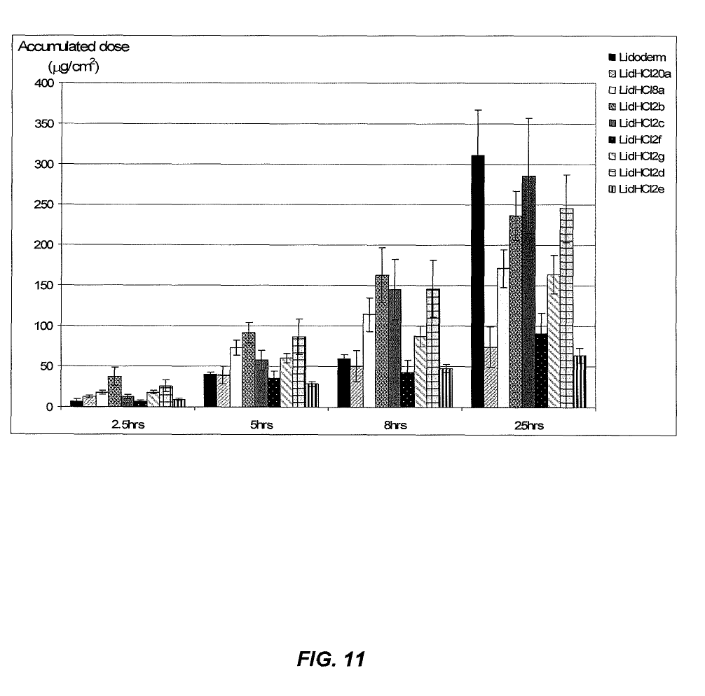

As shown in FIG. 11, the inclusion of AMP in the formulation led to a sharp increase in the flux (LidHCL8a vs LidHCL20a). FIG. 11 also shows the results of formulations in which the water was increased to ˜60%, considerable higher than the 35% present in the L504 lidocaine base formulation.

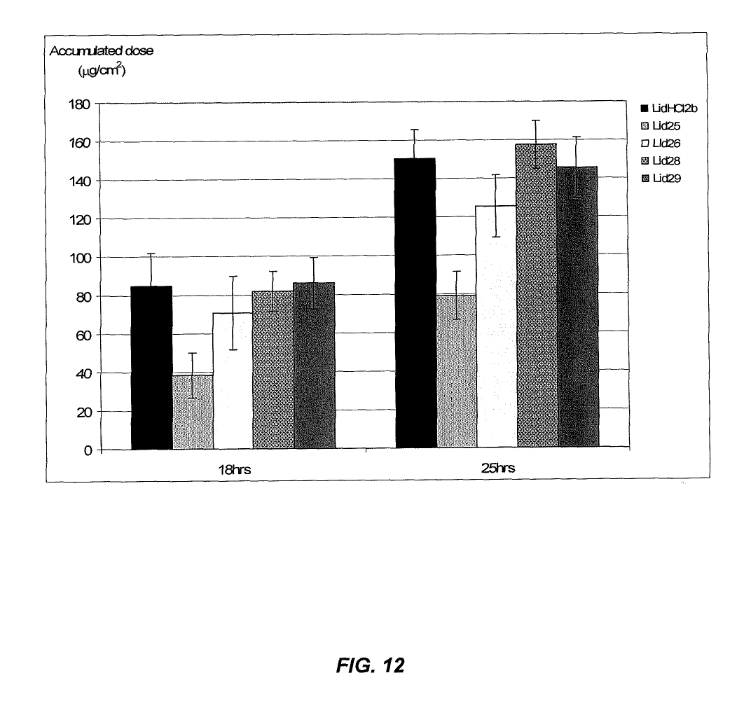

FIG. 12 shows the surprising result that when ethanol was removed from the solution and PG was increased, there was no undue loss to flux.

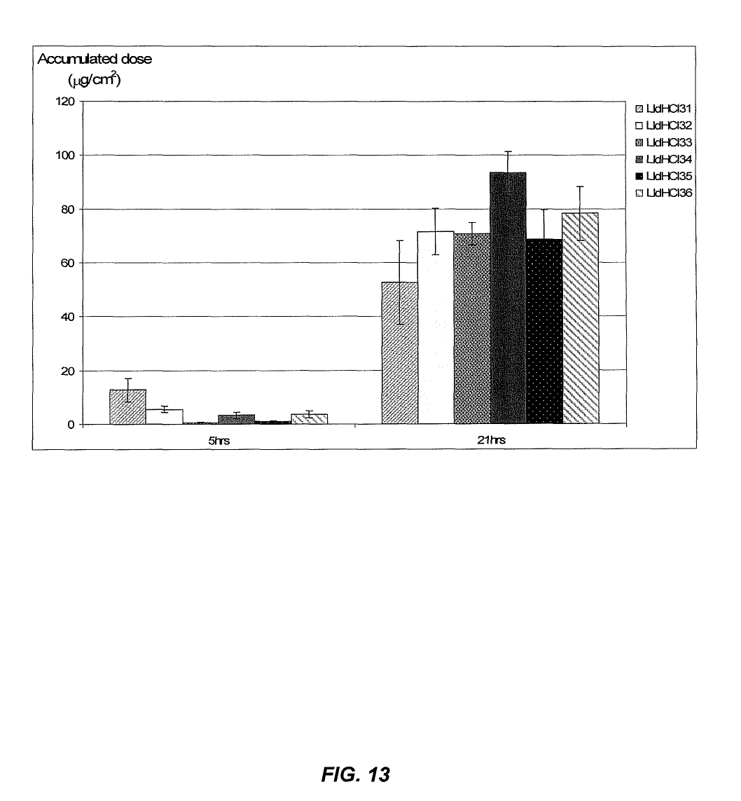

FIG. 13 shows the results of lidocaine HCL formulations with AMP and other molecular penetration enhancers. Specifically, molecular penetration enhancers in conjunction with AMP showed additional increase in flux. For example, the addition of Tw20 showed enhancement in flux over the base AMP formulation (LidHCL32 vs. LidHCL31).

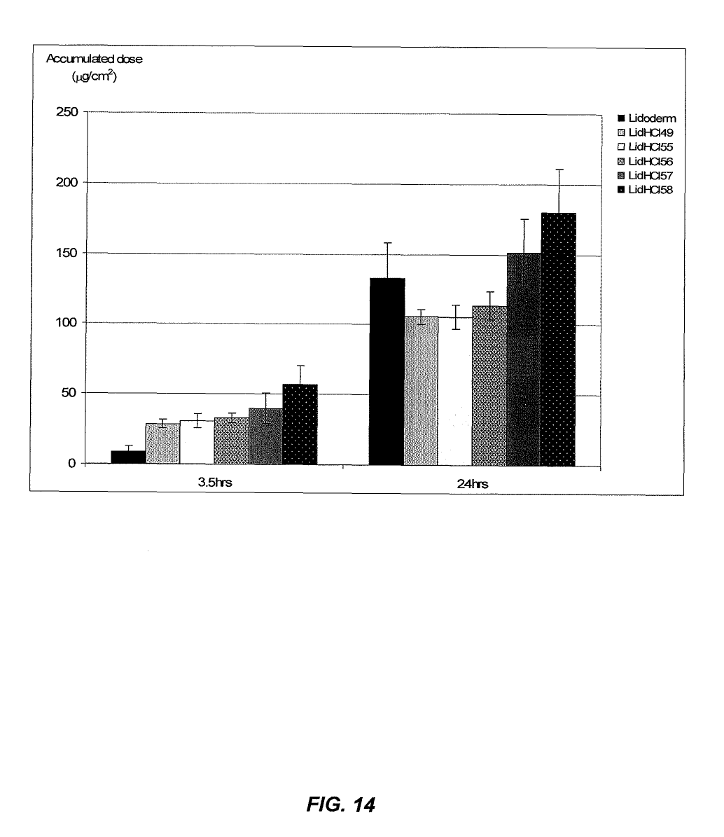

In order to mitigate the likelihood of stinging and irritancy, various formulations were tested with lower concentrations of PG. FIG. 14 shows the results using PEG300 as a substitute for part of the PG. Surprisingly, there was no significant drop in flux in the formulations with lower PG.

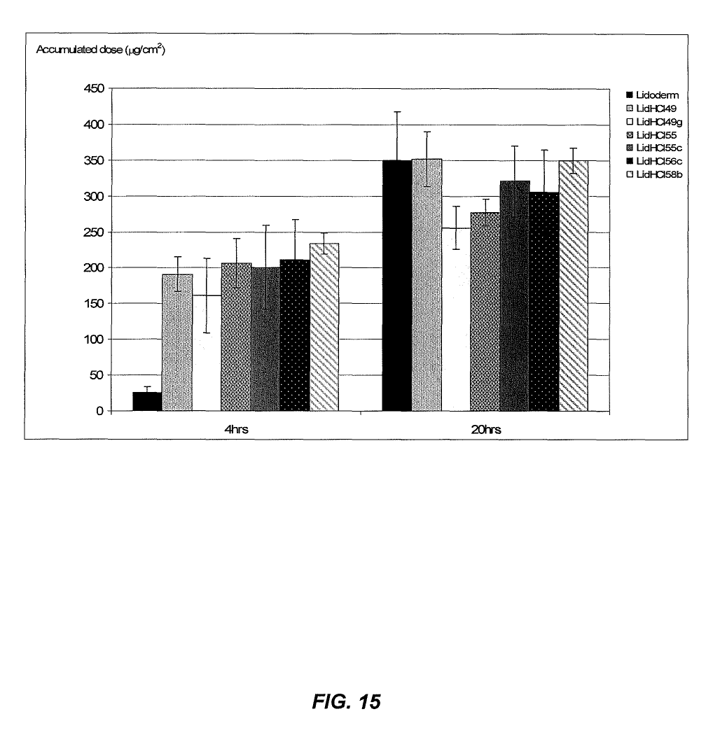

Various formulations were thickened with a hydroxypropyl cellulose or hydroxypropylmethyl cellulose to determine if such modifications affected the lidocaine flux. As can be seen in FIG. 15, there was no noticeable drop in flux when the viscosity of the formulation was increased.

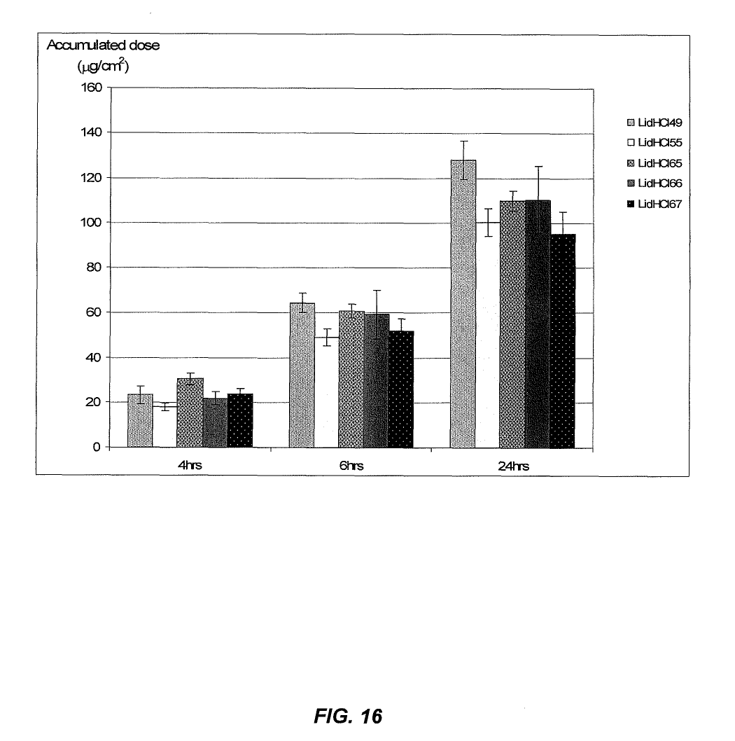

FIG. 16 shows the results of another variation to the AMP containing formulation. Specifically, the addition of EthAce to the AMP formulations demonstrated a small increase in the flux rate of lidocaine.

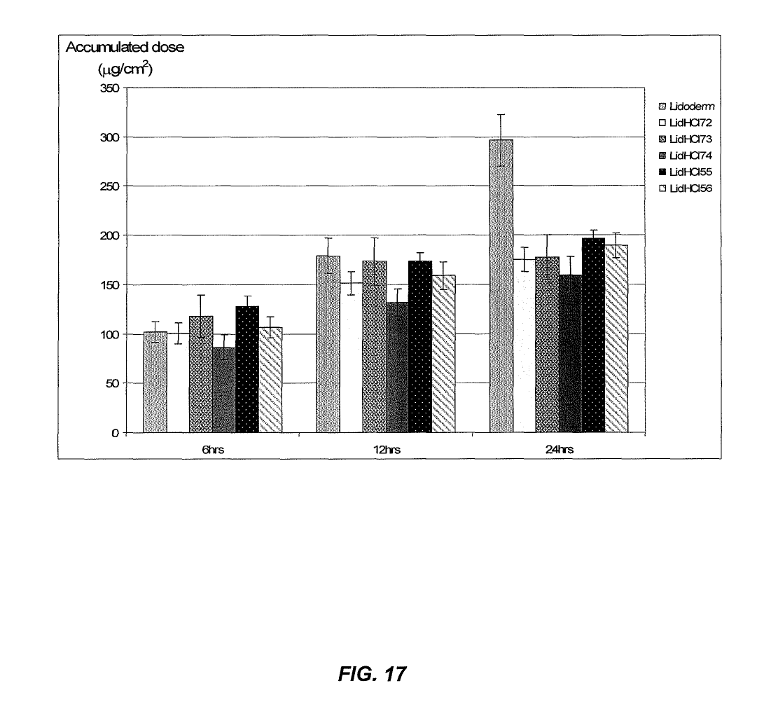

Further refinements to the AMP formulations were made in order to reduce the potential for the formulations to cause skin stinging and irritancy of the formulations. Specifically, various formulations were made to maximize the water concentration and minimize the EtOH (<3%) and PG (<25%) concentrations. PEG300 was added to prevent crystallization of lidocaine HCl in solution. The results are shown in FIG. 17.

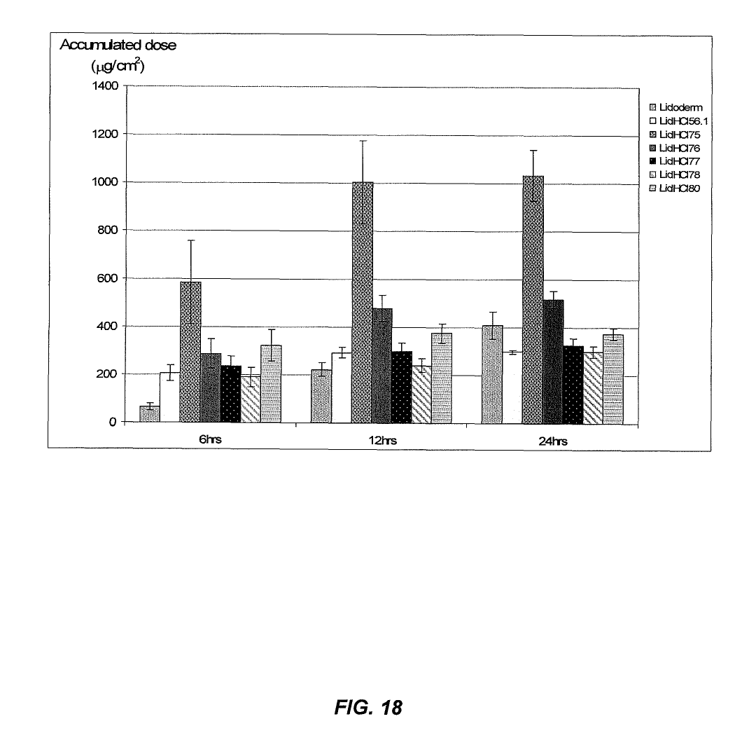

Other variants to the AMP formulation were tested in the low solvent vehicle. As can be seen in FIG. 18, the flux rates were similar. FIG. 18 also shows that the flux rates increase when the concentration of lidocaine HCl in the formulation is increases.

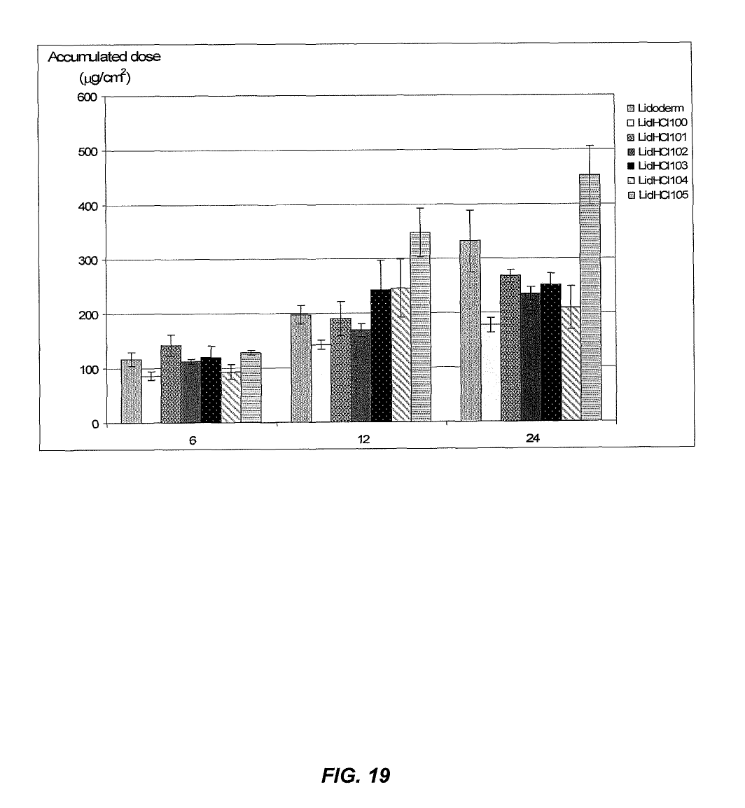

The formulations were further varied by adjusting the pH. Specifically, the pH of the solution was adjusted with NaOH to pH 6-7. Water concentrations were increased to ˜60% with minimal EtOH (1.5%) and PG (15%) in solution. FIG. 19 shows that the flux remained comparable to the comparator Lidoderm™.

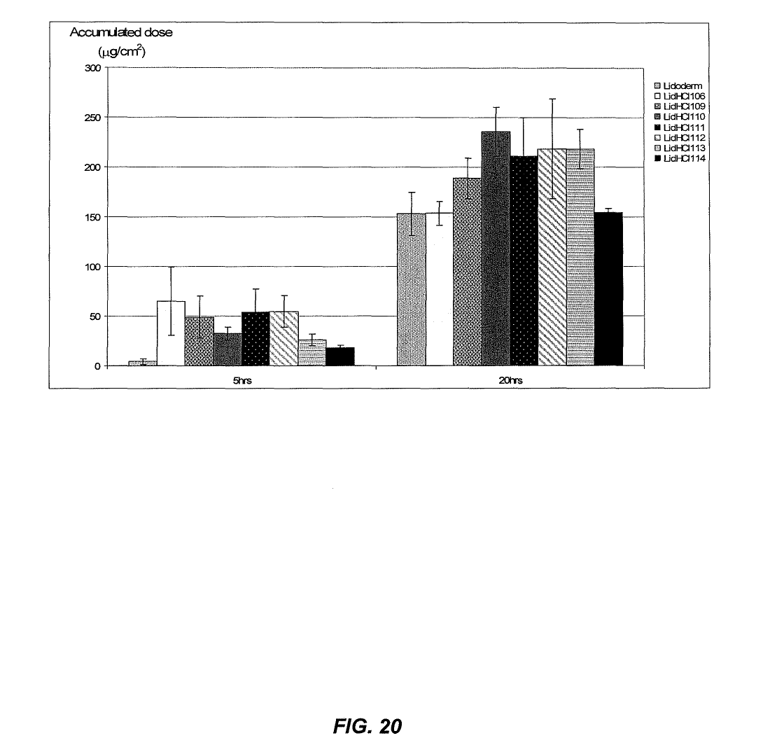

FIG. 20 shows other variations to the formulation. Specifically, the water concentration was maintained at 60%, with minimal addition of ethanol. PG was set at ˜15%. Addition of mild molecular penetration enhancers (such as LL) demonstrated a small increase in flux. In all cases, the flux of the formulations was approximately comparable to the control (Lidoderm™).

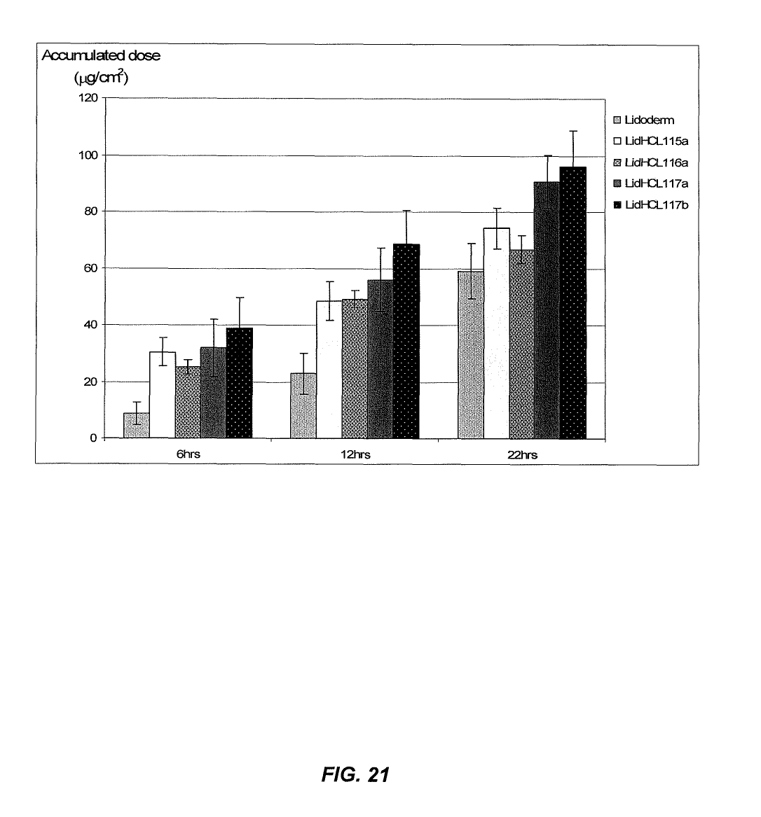

Another variation to the formulation is shown in FIG. 21. The water concentration was maintained at ˜60%, and ethanol was removed entirely from solution. PG and PEG300 were set at ˜15% and 10%, respectively. Addition of mild molecular penetration enhancers (Tw20 or SLSA) showed and increase in flux over the base pH adjusted formula.

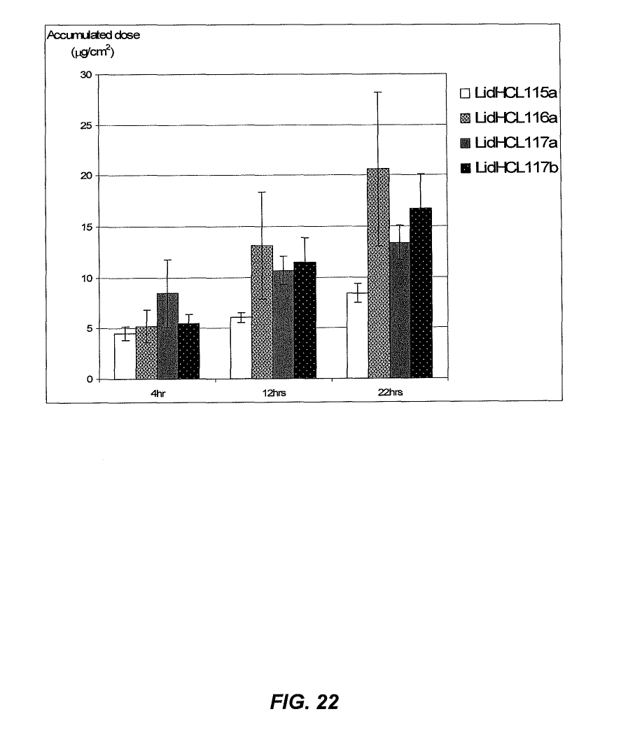

FIG. 22 shows that mild molecular penetration enhancers (Tw20 or SLSA) are able the increase the lidocaine flux from the formulation. These studies were carried out using shed snake skin as the substrate membrane. Shed snake skin is a more highly keratinized membrane than cadaver skin and is indicative of the flux expected across the top layer of the stratum corneum.

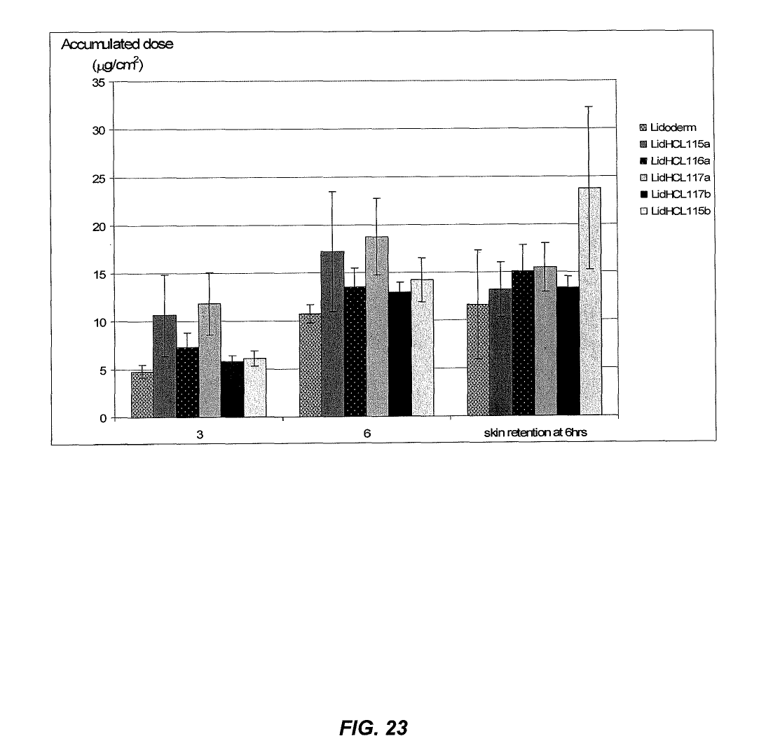

FIG. 23 depicts the results of further studies that show that an increase in flux is demonstrated when using mild molecular penetration enhancers (Tw20 or SLSA). These studies were carried out using cadaver skin as the substrate membrane.

### Example 3

### Methodology to Prepare the Formulations

### Preparation of Lidocaine HCL Solution Formulation (LidHCl 115a)

### Composition:

TABLE 3

| | | |
|---|---|---|
| Lidocaine HCl | 10% | w/w |
| Propylene Glycol | 16.5% | w/w |
| PEG300 | 10% | w/w |
| Water | 63.3 | w/w |
| Propyl Paraben | 0.1% | w/w |
| Methyl Paraben | 0.1% | w/w |

| | |
|---|---|
| 5N NaOH: | adjust pH to 6.0 |

### Procedure:

1. Combine propylene glycol, and PEG300.
2. Add methyl and propyl paraben. Mix thoroughly until the parabens are completely dissolved. Heating the solution to 60° C. will facilitate this process.
3. Add water to the mixture.
4. Add lidocaine HCl to the mixture while stirring.
5. After lidocaine HCl is fully dissolved, adjust the pH to 6.0 by dropwise adding 5N NaOH. Approximately 0.4 wt % NaOH is needed.
   NOTE: Dropwise addition of 5N NaOH will cause localized crystallization of lidocaine, which will disperse after stirring. For scale-up, it maybe preferable to add less water than 63.3% and qs appropriately with a more dilute NaOH solution.

### Preparation of Lidocaine HCL Solution Formulation (LidHCL117b)

### Composition:

TABLE 4

| | | |
|---|---|---|
| Lidocaine HCl | 10% | w/w |
| Propylene Glycol | 16.5% | w/w |
| PEG300 | 10% | w/w |
| Polysorbate 20 | 6% | w/w |
| (Tween 20 ™) | | |
| Water | 57.3% | w/w |
| Propyl Paraben | 0.1% | w/w |
| Methyl Paraben | 0.1% | w/w |

| | |
|---|---|
| 5N NaOH: | adjust pH to 5.9-6.1 |

### Procedure:

1. Combine propylene glycol, and PEG300.
2. Add methyl and propyl paraben. Mix thoroughly until the parabens are completely dissolved. Heating the solution to 60° C. will facilitate this process.
3. Add 95% of the water to the mixture.
4. Add Tween 20 to the mixture. Stir gently to prevent foaming of the Tween 20.
5. Add lidocaine HCl to the mixture while stirring gently.
6. After lidocaine HCl is fully dissolved, adjust the pH to 6.0 by dropwise adding 5N NaOH. Approximately 0.4 wt % NaOH is needed.
7. qs with remaining water.
   NOTE: Dropwise addition of 5N NaOH will cause localized crystallization of lidocaine, which will disperse after stirring. For scale-up, it may be preferable to add less water than 57.3% and qs appropriately with a more dilute NaOH solution.

### Example 4

### Stinging Protocol

Topical formulations, particularly those that are to be applied to diseased or damaged skin (e.g. cracks, fissures, open blisters, rash, and the like) may produce the sensory perception of stinging, a distinct discomfort to the user. A stinging test can be used to assess whether the novel topical formulations described herein produce a sensory perception of stinging.

For example, the study is designed to assess the sting potential of four topical formulations using a modification of a lactic acid sting assessment method. The test formulations are evaluated on skin that has been partially damaged (e.g. partial removal of the stratum corneum by tape stripping) to simulate diseased skin. Both a positive control (70% isopropyl alcohol) and a negative control (water) can be included to ascertain each subject's ability to sense the stinging sensation.

After providing informed consent, each subject receives a single dose exposure of 5 μL/cm² (40 μL/site) of a test formulation to an 8 cm² (2 cm×4 cm) surface abraded test site on their forearms (3 sites/arm), for a 10 minute duration. Skin abrasion is produced by repetitive tape stripping until a TEWL (Trans-Epidermal Water-Loss) measurement of 30 g/m²/hr, or greater, has been achieved (e.g. tape stripping will be performed 15 times followed by a TEWL measurement. If the TEWL is <30 g/m²/hr, 10 more strips will be collected, if TEWL is still <30 g/m²/hr, 10 more tape strip will be collected).

Subjects rated stinging, pain and discomfort at the site using a 100 mm visual analogue scale (VAS), one for each individual sensation, immediately after dosing and at 2, 5 and 10 minutes following topical application. Subjects provided a description of the sensations experienced following application of each test article. The subjects responses, measured in mm, were tabulated for each post-dosing assessment for each test sited.

The results are recorded and analyzed.

TABLE 5
Subjective Stinging Scores

| | | | | Mean Positive |
|---|---|---|---|---|
| | | Mean Treated | Mean Negative | Control score |
| Time after | | Score | Control Score | (70% |
| application | n | (LidHCL115a) | (Water) | Isopropanol) |
| Immediate | 10 | 1.44 | 2.45 | 10.84 |
| 2 Minute | 10 | 5.85 | 3.91 | 3.31 |
| 5 Minute | 10 | 1.74 | 3.01 | 1.87 |
| 10 Minute | 10 | 1.23 | 1.70 | 1.00 |
| Maximum score | 10 | 5.91 | 4.80 | 12.13 |

The results in Table 5 indicate that for most time points (the exception being t=2 minutes), the inventive formulation has a lower stinging score than the water application. There was one outlier at t=2 min for the inventive formulation. This outlier bumped up the average VAS score at this time point from approximately 2.5 to 5.85. Without this outlier, the inventive formulation has a lower stinging score than water at all time points. The positive control (70% isopropanol) follows a predictable pattern where the immediate stinging response is severe, followed by a rapid fall in pain. The LidHCL117b had 6% Tween 20, which lead to increased stinging over the LidHCL115a base formulation in the stinging test.

Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, whether or not such alternate, interchangeable, and/or equivalent structures, functions, ranges, or steps are disclosed herein, and without intending to publicly dedicate any patentable subject matter.

All publications, patents and patent applications referred to herein are incorporated by reference in their entirety to the same extent as if each individual publication, patent or patent application was specifically and individually indicated to be incorporated by reference in its entirety.

1-74. (canceled)

75. A method for alleviating pain associated with a Herpes zoster infection, said method comprising applying to an affected area an aqueous composition comprising a topically acting anesthetic active ingredient in a subanesthetic amount.

76. The method of claim 75, wherein said anesthetic active ingredient is selected from the group consisting of tetracaine, lidocaine, prilocalne, benzocaine, bupivacaine, mepivacaine, dibucaine, etidocaine, butacaine, cyclomethycaine, hexylcaine, proparacaine, lopivacaine and pharmaceutically acceptable salts thereof.

77. The method of claim 76, wherein said anesthetic active ingredient is selected from the group consisting of lidocaine hydrochloride and lidocaine base.

78. The method of claim 75, wherein said anesthetic active ingredient is in the amount of about 10% to about 20% w/w.

79. The method of claim 78, wherein said anesthetic active ingredient is in the amount of about 5% to about 10% w/w.

80. The method of claim 75, wherein the composition further comprises a molecular penetration enhancer.

81. The method of claim 80, wherein said molecular penetration enhancer is a polyhydric alcohol.

82. The method of claim 75, wherein the method of application of the composition is by spraying.

83. The method of claim 75, wherein the aqueous composition is a non-stinging and non-irritating pharmaceutical composition.

84. The method of claim 80, further comprising an additional molecular penetration enhancer.

85. The method of claim 84, wherein said additional molecular penetration enhancer is a member selected form the group consisting of polysorbate 20, methyl laurate, isopropyl palmitate, N-methyl-2-pyrrolidone, aminomethylpropanol, 1,2,6-hexanetriol, methyl salicylate, myristyl lactate, sodium lauryl sulfoacetate and a combination thereof.

86. The method of claim 85, wherein said additional molecular penetration enhancer is in an amount of about 1% to about 10% w/w.

87. The method of claim 85, wherein said additional molecular penetration enhancer is polysorbate 20 w/w.

88. The method of claim 87, wherein polysorbate 20 is present in an amount of about 5% to about 10% w/w.

89. The method of claim 75, wherein the pH of the composition is between about 4 and about 8.5.

90. The method of claim 75, wherein the pH of the composition is between about 5.5 and about 7.

91. The method of claim 75, wherein said composition is substantially free of a lower alkanol.

92. The method of claim 75, wherein said composition further comprises a preservative or is sterile.

93. The method of claim 75, wherein said composition is used for the management of pain associated with a Herpes zoster infection.

94. The method of claim 75, wherein said Herpes zoster is acute Herpes zoster.
